(19) United States
(12) Patent Application Publication
(10) Pub. No.: US 2018/0214229 A1
(43) Pub. Date: Aug. 2, 2018

(54) SHIELDING DEVICES

(71) Applicant: CONCEPTOMED AS, Ballstad (NO)

(72) Inventors: Christian MIDE, Ballstad (NO); Marius ANDRESEN, Oslo (NO); Rolf BLOMVÅGNES, Rong (NO); Kevin GEERS, Oslo (NO); Kine Therese Reppe ARNTZEN, Gravdal (NO); Johanna Lisa Marie HENRIKSSON, Sundsvall (NO)

(21) Appl. No.: 15/886,998

(22) Filed: Feb. 2, 2018

Related U.S. Application Data
(63) Continuation of application No. 14/432,506, filed on Mar. 31, 2015, now abandoned.

(30) Foreign Application Priority Data
Oct. 5, 2012 (GB) 1217898.4
Jan. 31, 2013 (EP) 13153547.8
Jul. 24, 2013 (GB) 1313229.5

(51) Int. Cl. A61B 46/27; B65H 35/08; B42C 19/02; A61M 5/52; A61B 90/00; A61M 25/02; A61B 5/15; A61B 46/20

(52) U.S. Cl. CPC A61B 46/27; B65H 35/08; B42C 19/02; A61M 5/52; A61M 2205/59; A61M 25/02; A61B 5/150839; A61B 90/05; A61B 2046/205; A61B 90/00

(57) ABSTRACT

A device for shielding at least part of a limb from the view of a wearer of the device. The shielding device may include a shield with a first configuration in which it is substantially planar and a second configuration in which it has a three-dimensional shape. The shield may include one or more hinge lines, enabling it to be converted from the first configuration to the second configuration. The device also may include an attachment portion with one or more elongate members to attach around a limb of a wearer in use. An edge portion of the shield may include an outer edge opposite the hinge line that is at least partially divided into independently moveable sections such that the edge portion can be flexed to help convert the shield into a three-dimensional configuration.

### RELATED APPLICATIONS

The present application is a continuation of U.S. application Ser. No. 14/432,506, filed on Mar. 31, 2015. U.S. application Ser. No. 14/432,506 is incorporated herein by reference in full.

### TECHNICAL FIELD

In a clinical setting or anywhere else where a patient is being treated by a medical professional, it is important for the patient to feel at ease. Many medical procedures involve the use of needles or other devices on a patient's body. Often patients are afraid of needles, or feel discomfort at the sight of medical procedures or injuries. Some patients may even faint at the sight of needles, blood or an open wound. While patients may close their eyes, or look away, during an injection or other medical procedure, this prevents the medical professional(s) involved from maintaining eye contact with them. It is desirable for a patient to be able to see and speak to medical professionals while he or she is being treated, but without risk of viewing the procedure that is being carried out or the site of an injury.

### BACKGROUND OF THE INVENTION

It has previously been proposed to distract patients, especially children, during intravenous injections using a board with an opening through which a child can insert his/her arm. In U.S. Pat. No. 5,044,378 such a distraction board shows a clown's face with an opening at its mouth and features such as a squeezable nose to distract the child while an injection is administered on the other side of the board. US 2006/0272651 discloses a diversion board that includes an aperture for an arm or body limb to fit through. Such boards are designed to stand on a horizontal surface, for example a table in a doctor's office, clinic or hospital.

While diversion boards can block the view between a patient and a medical procedure such as an injection, they have several drawbacks. The boards may be bulky and difficult to move and/or store between uses. When a diversion board is re-used for different patients, it needs to be cleaned effectively and there is a risk of cross-contamination. A particular problem is that the aperture in the board must be large enough to accommodate a range of different sized limbs and the board must also be large enough to block the field of vision when used to shield different parts of the body (arm, leg, etc.). Such boards may not therefore be suited for use with a range of different patients—children and adults—or for a range of different medical procedures. In fact, it may often be that the board prevents a patient from viewing the injection or other procedure but also makes it difficult for a medical professional to see over or around the board to maintain eye contact with the patient.

There remains a need for effective shielding devices and methods for use during medical procedures (and any other procedures where a subject may be distressed by the sight of the procedure).

### SUMMARY OF THE INVENTION

According to a first aspect of the present invention there is provided a device for shielding at least part of a limb from the view of a wearer of the device, the device comprising a self-standing shield portion and an attachment portion, the attachment portion comprising an elongate member to attach around a limb of a wearer in use.

It will be appreciated that such a shielding device can be attached around a limb of a wearer (e.g. a patient) rather than the patient needing to insert a limb through a fixed aperture in a diversion board. This makes it easier for the device to be applied, especially in situations where a patient is injured and unable to move the limb without causing pain. The elongate member can be easy to manipulate so that the wearer or another person is able to wrap it around the limb to be shielded from view. The device is therefore ideal for being applied without the wearer needing to lift or move the limb. The elongate member may not wrap completely (i.e. 360°) around a limb, but around a portion of its circumference. The elongate member is preferably dimensioned so as to attach around at least 50%, 60%, 70%, 80%, 90% or even up to 100% of the circumference of a limb.

The functions of the device are split between a portion designed for shielding and a portion designed for attachment, which can make it much more flexible in its range of use. For example, the device may be worn not only to prevent a patient from viewing an injection but also to shield a wound or broken limb from view and even to protect the site of injuries. As the shielding device is attached around a limb, it can be worn even if the patient moves around, for example carried on the limb as the patient is transferred into an ambulance or walks around a hospital. The self-standing shield portion has sufficient rigidity that it stands up from the limb regardless of the orientation of the limb e.g. even if the wearer moves around.

The attachment portion carries out the function of attaching the self-standing shield portion to a limb. By providing an elongate member to attach around the limb, the shield portion does not need to extend on all sides of the limb (e.g. as compared to a diversion board comprising an aperture for a limb to pass through). The elongate member can wrap closely around the limb to enlarge the field of view outside the shield portion. Furthermore the attachment portion can be designed to adapt to different limbs. The shield portion may have a standard size, that is large enough to block a patient's view of the limb to which it is attached but not so large as to prevent a medical professional behind the device from looking over or around the shield portion to have eye contact with the patient. Meanwhile the attachment portion is preferably adjustable e.g. to accommodate different sizes and types of limb. As will be discussed in more detail below, the one or more elongate members of the attachment portion can be designed to provide this adjustability. Advantageously, the shielding device is useable with a range of patients (children and adults) and/or with a range of different limbs (upper or lower extremities; necks, arms, wrists, hands, legs, ankles, feet, etc.).

The self-standing shield portion has rigidity to stand regardless of any adjustment of the attachment portion. The shield portion can stand at any angle θ from the limb, where 0<θ<180°. Preferably the shield portion is self-standing, in use, at an angle θ from the limb, where 0<θ<90°. This ensures that the shield portion is angled from the limb so that a medical professional can access the shielded site to carry out a procedure without being hindered by the device, while the wearer can view the other side of the shield portion. The side of the shield portion that faces a wearer in use preferably has a display function, e.g. printed with graphics and/or text or even comprising a display screen, to distract or educate the wearer. The shield portion is preferably made of a lightweight (albeit rigid) material e.g. cardboard, plastic, or laminates thereof. The shield portion is preferably not made from a soft material such as a fabric or cloth-like material. Such materials may also pose a hygiene risk as they may be more easily contaminated than substantially non-porous materials such as paper, card or plastic.

In one set of embodiments, the shield portion may be separable from the attachment portion. The shield portion may be removably connected to the attachment portion in use. This provides flexibility for the shielding device, as different shield portions may be selected for use with a given attachment portion, or vice versa. In one example the shield portion chosen for an infant wearer may be colourful or carry an illustration while a shield portion chosen for an adult wearer may be plain. In another example the same shield portion may be used for different patients, but with a different attachment portion chosen depending on the size and/or type of limb around which the device is to be attached. This is not withstanding that the attachment portion is preferably adjustable.

Another advantage of the shield portion and attachment portion being separable is that one portion or the other may be discarded after use while the other portion is re-used. The risk of spreading infection can be reduced by disposing of a contaminated portion of the device. On the other hand, environmental impact can be reduced by cleaning/sterilising and re-using a portion of the device where possible. The shield portion and the attachment portion can be made of different materials.

In another set of embodiments, the shield portion and attachment portion may be integrally provided together as a shielding device. For example, the shielding device may be formed from a single piece of material. As will be described in more detail below, forming the device from a single piece of material is advantageous as it makes it much simpler to manufacture, and package, multiple devices that can be conveniently dispensed one-by-one. The single piece of material may be shaped to provide a bulk portion as a shield and one or more elongate portions for attachment around a limb. The shielding device may be intended for multiple uses, for example with wipeable surfaces that can be cleaned between uses. Or the shielding device may be intended for single use, for example provided with instructions for disposal after use. This can avoid any risk of infection being spread amongst patients.

The reusability of the shielding device, or at least of the shield portion, may inform the material(s) and/or construction used. However, for the shield portion to be self-standing the material(s) and/or construction must provide a certain level of stiffness or rigidity. In one set of embodiments the shield portion may be substantially two-dimensional in construction, for example a planar piece of material. A substantially two-dimensional shield portion may be made from relatively stiff or thick materials, for example cardboard (rather than paper), plastics, or even a lightweight metal. A shield portion made of metal could be sterilised in an autoclave for re-use.

In another set of embodiments the shield portion may be substantially three-dimensional in construction. A three-dimensional shape can help the device to effectively block the field of view around a site where there is an injury or a medical procedure is to take place. However the Applicant has recognised that another benefit of forming the shield portion in a three-dimensional shape is that this can increase its stiffness so that it is better able to be self-standing. The shield portion may therefore be more stable when it has a three-dimensional construction, especially when it is made from a relatively thin piece of material such as a sheet of paper or card. The Applicant has recognised that such a shield portion can be made from relatively flexible or thin materials, such as paper, but still be self-standing by virtue of its three-dimensional shape.

This feature is considered novel and inventive in its own right, and thus when viewed from a second aspect the present invention provides a device for shielding at least part of a limb from the view of a wearer of the device, the device comprising: a shield portion having a first configuration in which it is substantially planar and a second configuration in which it has a three-dimensional shape, the shield portion comprising one or more hinge lines enabling it to be converted from the first configuration to the second configuration; and an attachment portion, the attachment portion comprising one or more elongate members to attach around a limb of a wearer in use.

It is a benefit of this aspect of the invention that the shield portion can be made from less stiff, thinner, lighter and/or cheaper materials than a self-standing shield portion, for example paper or thin card instead of cardboard or plastic. The shield portion, and preferably the entire shielding device, may be made a sheet of paper or card that is about 1 mm thick. Such a shielding device (or at least the shield portion thereof) may therefore be better suited as a single use device that is intended to be discarded after use. As well as the potential for a lower material cost, the shield portion may more easily be crumpled or compacted for disposal. Even so, the shielding device (or at least the shield portion thereof) may be re-used where desired or necessary. For example, a thin and flexible but wipeable material such as waxed paper or plastic laminate may be used for the shield portion.

A shield portion that is planar in its first i.e. initial configuration and preferably thin can generally be printed on more easily than a self-standing shield that may be relatively thick or even have a three-dimensional shape. In some embodiments it is therefore preferable that (at least) the shield portion is printed upon. Graphics and/or text may be printed on the shield portion, on at least one side or on both sides. For a shielding device intended to distract children during medical procedures there may be cartoons or the like printed on the shield portion. In other examples the shield portion may be printed with advertising or brand information for a medical product or service supplier. Information relating to use of the device and/or instructions for use may be printed on at least one side.

The hinge line(s) may be any line of weakness in the material of the shield portion. If the shield portion is made of plastic, for example, then the hinge line(s) may be provided by thinning or perforating the plastic to form a living hinge. If, for example, the shield portion is made of cardboard or paper then the hinge line(s) may be scored or perforated lines. The hinge line(s) enable the shield portion to be folded or creased into the second configuration having a three-dimensional shape. In one set of embodiments the hinge line(s) are bi-directional, e.g. being perforated through the material rather than scored on one side, to enable the shield portion to be converted into a second configuration either into or out of the plane of its first configuration. This provides the flexibility of converting the shield portion into a concave or convex configuration. If there is a difference in the text and/or graphics displayed on either side of the shield portion, then this allows a user to select what will be displayed to the wearer.

The three-dimensional shape of the second configuration may be angular and/or curved. Each hinge line may itself be linear and/or curved. The number and/or arrangement of the hinge line(s) can be provided to dictate the three-dimensional shape that is formed in the second configuration. As different shapes may be appropriate for shielding different types of limb, different locations, different medical procedures or injuries, etc. then a shield portion with a particular arrangement of hinge line(s) may be designated for one or more particular uses. In its second configuration the shield portion is able to block the field of view of the wearer. This may be achieved by the shield portion covering over the site of a wound or the like, e.g. in a domed configuration. However it is preferable for the three-dimensional shape to be self-standing. This can ensure that medical professionals have space behind the shield portion to carry out procedures without the shield portion being in the way.

Preferably the shield portion comprises at least one curved hinge line that enables it to be converted to its second configuration. As well as helping to convert the shield portion into a three-dimensional shape that is preferably curved, such a hinge line can advantageously allow the folded edge of the shield portion to substantially conform to the curved shape of a limb. The shield portion may therefore be self-standing with a curved edge that follows the contour of a limb. This can help to ensure that the shield portion stands stably on the limb, preferably at an angle θ from the limb that satisfies 0<θ<90°, and preferably without a gap between the shield portion and the limb that could allow the wearer to catch sight of the procedure being performed. A further advantage of a curved edge is that the device can be rotated around the limb easily, and without causing any pain or discomfort. As is mentioned above, the curved hinge line(s) may be physically formed in the device, e.g. as a perforated or scored line, so as to ensure that the shield portion folds along the designated hinge line(s) when it is converted between its first and second configurations.

As well as dictating the three-dimensional shape of the shield portion, at least one hinge line is preferably used to define an edge portion, which forms a folded edge of the shield portion when it is converted into the second, three-dimensional configuration. The edge portion preferably has a folded edge defined by the hinge line. This folded edge may rest against the limb of a wearer when the device is attached, so the rest of the shield portion stands up at an angle from the limb. As is mentioned above, if the hinge line in the shield portion is curved, rather than straight, then the folded edge can wrap around a limb and provide for a wider shielding angle. This may also enable the device to be easily rotated around the limb to shield the wearer's view. The Applicant has recognised that the shape of the edge portion can also be important for ensuring that the device is comfortable to wear. It is not necessary for the edge portion to follow the shape of the underlying limb. However, it is preferable for the edge portion to be curved so that it is more likely to conform to a limb and any garment (or other foreign body) changing the geometry of the underlying limb. If the device is cut out from a relatively rigid sheet material, rather than being formed of a completely flexible e.g. fabric material, then the outer edge could be sharp and there is a risk of cutting the wearer's skin. If the edge portion has a curved outer edge then this edge is less likely to press against the wearer's skin when the edge portion is folded away from the rest of the shield portion in its second (3D) configuration. It is therefore preferable that the edge portion comprises an outer edge opposite the hinge line that substantially follows the hinge line. Where the hinge line is curved then the outer edge is preferably also curved to follow the hinge line.

The shape e.g. curvature of the hinge line may be chosen so that the folded edge will tend to follow the outline of a limb when the shield portion is converted to its second, three-dimensional configuration. However the rigidity of the edge portion may limit the curved shape that can be achieved. In a particularly preferred set of embodiments, the edge portion is shaped so that it can flex and conform more easily to a wearer's limb. This may be especially helpful where the device, or at least the shield portion, is formed of a sheet material such as a sheet of paper or thin card that is limited in its flexibility. It is therefore preferable that the edge portion comprises an outer edge opposite the hinge line that is at least partially divided into spaced sections such that the edge portion can be flexed to change the length of the outer edge. When the device is folded into its three-dimensional configuration, this edge portion can be flexed to wrap around a wearer's limb with a variable outer edge. Preferably the spaced sections are arranged substantially equally along a path that follows the hinge line. As the edge portion has a number of sections, it can be gently moved and rotated around the limb more easily with decreased likelihood of cutting the skin.

In one example, the outer edge may be divided into spaced sections separated by notches extending substantially perpendicular to the hinge line. In one example the spaced sections may extend substantially perpendicular to the hinge line, e.g. in the form of teeth. This has the benefit that the teeth are able to spread as the device is arranged on a limb, increasing the stability of the shield portion and preventing it from falling forward. With an outer edge that is divided into sections, the device is able to be fastened much more tightly to the limb, allowing it to act as a tourniquet due to the increased freedom in the shape of the edge portion. As there are gaps between the sections, the edge portion can be pulled tighter without any extra tension on the shield portion. Preferably the edge portion is arranged between the elongate members of the attachment portion. This allows pressure to build up distal to the shield portion, increasing the ease of procedures carried out, for example during venipuncture when establishing intravenous ports.

In one set of embodiments the spaced sections may simply comprise undulations in the outer edge. Spaced sections in the form of a wavy edge may not greatly increase the flexibility of the edge portion but may at least act to blunt the outer edge, so that it is less likely to cut into the skin if the shield portion is rotated around a wearer's limb.

An alternative way to reduce the rigidity of the edge portion is for the edge portion to comprise an outer edge opposite the hinge line that is at least partially divided into sections, not necessarily spaced sections, such that the edge portion can be flexed to change the length of the outer edge. Preferably the sections are independently moveable. The edge may be divided into sections by cuts, for example cuts substantially perpendicular to the edge. The sections can move apart, like a fringe, to soften the outer edge so that it is less likely to cut into a wearer's skin. Because the sections are free to move relative to one another, this makes the edge portion more flexible and better able to adapt to the shape and/or curvature of a limb. The flexibility of the edge portion can allow it to change its curvature as the shield portion is converted between its first and second configurations, even assisting the shield portion in adapting a three-dimensional shape in the second configuration.

The Applicant has realised that an edge portion divided into sections can be flexible enough that it allows the shield portion to be converted between its first and second configurations even without any hinge line(s) being provided. This may especially be achieved if the shield portion is formed integrally from a single piece of flexible material. This is considered novel and inventive in its own right. Thus when viewed from a third aspect, the present invention provides a device for shielding at least part of a limb from the view of a wearer of the device, the device comprising: a shield portion formed from a sheet of flexible material and having a first configuration in which it is substantially planar and a second configuration in which it has a three-dimensional shape; and an attachment portion, the attachment portion comprising one or more elongate members to attach around a limb of a wearer in use; wherein the shield portion comprises an edge portion having an outer edge that is at least partially divided into independently moveable sections such that the edge portion can be flexed to convert the shield portion from the first configuration to the second configuration.

It will be appreciated that the independently moveable sections allow the edge portion to flex and change its curvature e.g. by changing the length of the outer edge. Since the entire shield portion is formed from a sheet of flexible material, this change in curvature is not limited to the edge portion but also causes the shield portion to flex out of its first, planar configuration into one or more second, three-dimensional configurations. It is not necessary for the shield portion to include any hinge line(s) in order to convert between its first and second configurations. The changing shape of the edge portion due to the moveable sections can be sufficient to convert the shield portion from the first to second configuration. Although the edge portion may have any suitable shape, it is preferable for the edge portion to be curved, as this can help to ensure that the edge portion follows the curvature of a limb. It is desirable for the edge portion to lie flat against a limb so that a wearer can not seen underneath the shield portion while a procedure is being carried out.

In practice, the material of the edge portion may flex as the elongate members are wrapped around a limb. This may be assisted by the edge portion being arranged between the elongate members of the attachment portion. The attachment portion may be formed from one or more separate pieces of material that are attached to the shield portion. However, in a preferred set of embodiments the whole device is formed from the same sheet of flexible material, e.g. a single sheet of paper or card.

The independently moveable sections may be abutting, for example the outer edge may be divided into sections by cuts, for example cuts substantially perpendicular to the edge. In a set of embodiments the independently moveable sections are spaced apart. The spaced sections may extend substantially perpendicular to the outer edge, for example in the form of teeth. As is explained above, such spaced sections can easily spread to allow the edge portion, and the rest of the shield portion, to flex into a curved three-dimensional configuration.

It will be appreciated that at least in some embodiments the shield portion may comprise one or more hinge lines as well as an edge portion having an outer edge that is at least partially divided into independently moveable sections. Such embodiments may take advantage of the hinge line(s) providing control over the three-dimensional shape of the shield portion in combination with a fringed outer edge that can softly spread against the limb of a wearer so that the device is comfortable to wear and adjust.

As is described above, the shield portion can be separable from the attachment portion (for example using any of the features outlined earlier), but preferably the attachment portion is integral with the shield portion. The shielding device may therefore take the form of a single piece of material that can be converted from the first configuration to the second configuration. In a preferred set of embodiments the shielding device is made from a single sheet of paper or card. Such a shielding device is ideally suited as a disposable, single use item.

In a set of embodiments wherein the shield portion is integrally formed with the attachment portion, e.g. as a single piece of material, the device may be removed from a wearer after use by releasing the elongate member(s) that are attached around a limb of the wearer. This may be facilitated by the elongated member(s) being removably fastened, to one another or to the device, for example using a releasable adhesive or fastener such as Velcro™ However it may be preferable for the elongate member(s) to be permanently fastened when the device is attached, e.g. using a non-releasable adhesive, so as to ensure that the device does not become accidentally detached during use. So as to facilitate removal of the device, in one set of embodiments the device comprises at least one point of weakness to enable the shield portion to be separated from the attachment portion after use. The point(s) of weakness may comprise, for example, a perforation or notch in the piece of material. The point(s) of weakness may be located between the shield portion and the elongate member(s) of the attachment portion.

In addition to any hinge line(s), and optionally any point(s) of weakness as described above, the shielding device may comprise one or more perforated boundaries. This can make it easier for the device to be removed from a piece of material from which it is formed. A spare portion of material may be removably attached to the shielding device, for example a portion of material bearing information and/or graphics relating to the shield and/or its use(s). The spare portion may take the form of a guide, certificate, coupon, sticker, etc. for the wearer or for a medical professional. In at least some embodiments it is preferable for the spare portion to have a writeable surface. In some embodiments the spare portion of material may be defined between the shield portion and the elongate member(s) of the attachment portion, e.g. in the same sheet. In some embodiments the spare portion may be defined by a front or back cover sheet that is attached to the device e.g. folded in front or behind as another sheet. Of course a device may include more than one such spare portion.

A further advantage is that at least the shield portion, and preferably the whole device, can be easy to store, transport and handle manually in its planar configuration. If the shield portion or device is made of thin, lightweight sheet material such as paper then multiple shield portions or devices in a planar configuration can be stacked on top of one another in a particularly compact arrangement. In one set of embodiments there may be provided a plurality of shield portions or devices made of sheet material (e.g. paper) that are bound together in a stack. The stack may be bound e.g. by staples or adhesive to form a spine, or by a separate spine such as a plastic comb or metal coil. In other examples the stack may be bound together by an outer wrapper or other dispensing packet. It is preferable that the sheets can be removed from the stack one-by-one. This can provide a particularly convenient way of storing and dispensing shield portions or devices (typically intended for single use) that are formed from a single sheet of material e.g. paper. Furthermore such a stack may be used to dispense whole shielding devices where they are each made from a single piece of paper or other sheet material.

This feature is considered novel and inventive in its own right, and thus when viewed from a further aspect the present invention provides a dispenser of shielding devices that comprise a shield portion and an attachment portion, the attachment portion comprising one or more elongate members to attach around a limb of a wearer in use, the shielding devices each being formed from a single sheet of material, and the dispenser comprising a plurality of the shielding devices bound together.

It will be appreciated that such a dispenser may resemble a calendar, booklet or notepad with sheets that can be removed (e.g. pulled or torn out) one-by-one. It is preferable for the sheet material to be thin so that the dispenser is compact and can provide a large number of shielding devices in a relatively small volume. As is mentioned above, various sheet materials such as cardboard, thin plastic or even metal foil may be used, but preferably the shielding devices are each formed from a sheet of paper. If the sheet material is rigid enough for the shield portion of each shielding device to be self-standing then hinge line(s) may not be required. However, in keeping with preferred embodiments in which the sheet material is thin e.g. plastic or paper then it is also preferable for the shield portion of each shielding device to comprise one or more hinge lines and/or a flexible edge portion enabling it to be converted from a first configuration, in which it is substantially planar, to a second configuration in which it has a three-dimensional shape. It is further preferable for the three-dimensional shape to be self-standing. In some examples, the number and/or arrangement of hinge line(s) can provide a shield portion tailored for a particular use, as is described above. One or more curved hinge line(s) may be preferred to enable the folded edge of the shield portion to closely conform to a limb of a wearer. In other examples, the shield portion may be flexed into a curved three-dimensional shape by flexing an edge portion, preferably an edge portion having an outer edge that is at least partially divided into independently moveable sections.

In one set of embodiments the shielding devices are bound together in a pad or booklet. The pad may be tightly bound e.g. by staples or adhesive, so that it is most convenient to remove the top sheet at any time. In another set of embodiments the shielding devices are bound together in a stack or booklet of separated sheets. The shielding devices may be bound together by respective spines that extend along at least one edge of each device. Preferably each shielding device is removable from its spine. This means that a user can select which sheet to remove from the stack or booklet without disturbing the others. If different shielding devices have different sizes, shapes or information/graphics printed thereon then it may be desirable to pick one sheet rather than another for particular wearer and/or for a particular use.

For ease of manufacture, each device is preferably integrally formed with a respective spine. The device and its spine may share a perforated boundary, or other suitable line of weakness, to facilitate removal of the device from the spine. This boundary may extend along the top, bottom and/or side(s) of the device. In one set of embodiments, each device is partially cut along its boundary with the spine. Where the boundary extends along both the attachment portion and the shield portion, e.g. the spine extends along a side of the device, then it is preferable for each device to be partially cut along the length of an elongate member of the attachment portion. This can help to ensure that elongate member is not accidentally damaged when the device is pulled away from the spine.

When viewed from a further aspect the present invention provides a method of manufacturing a pad or booklet for dispensing shielding devices, comprising: forming each of a plurality of shielding devices from a single sheet of material; connecting the shielding devices together with a common spine; and forming a line of weakness to enable each shielding device to be dispensed from the spine. The method may further comprise partially cutting the sheet of material along the spine, e.g. so as to dictate which portion of the device is pulled away from the line of weakness. In a preferred set of embodiments, each shielding device comprises a shield portion and an attachment portion, the attachment portion comprising one or more elongate members to attach around a limb of a wearer in use. Each device may be partially cut along the length of an elongate member of the attachment portion. The line of weakness may be formed along at least one of the top, bottom and/or side(s) of the shield portion.

Another advantage of a shielding device being made from a single sheet of material e.g. paper is that it can be easy to manufacture. The shielding device can be cut or stamped to have a desired shape defining the shield portion and the attachment portion. Multiple shielding devices can be tessellated in a large sheet of material. Or in a run of material, the shielding devices may be provided end-to-end or side-by-side. The sheet material may be severed to form individual shielding devices. In other embodiments, the sheet material may be perforated at the boundary of the shielding devices. In yet other embodiments the sheet material may be folded, e.g. concertina-style, to form a stack of shielding devices. Such a folded stack may conveniently be used to form a bound pad of shielding devices. In all of these embodiments, a continuous manufacturing process is possible.

When viewed from a yet further aspect the present invention provides a method of manufacturing shielding devices from a sheet of material (such as paper), each shielding device comprising a shield portion and an attachment portion, the attachment portion comprising one or more elongate members to attach around a limb of a wearer in use, wherein multiple shielding devices are formed side-by-side in the same sheet of material. The method may further comprise the step of folding, weakening (e.g. perforating) or severing the sheet of material to separate the shielding devices from one another.

It will be appreciated that the shielding devices may be arranged laterally side-by-side or longitudinally side-by-side e.g. end-to-end. In the latter case, the shielding devices may be arranged adjacent to one another in a longitudinal direction and preferably overlapping such that a device sits between two adjacent devices. In one set of embodiments, the shield portion of each device is shaped such that it fits between the elongate legs of an adjacent device. In an arrangement such as this, a plurality of devices can be produced from the same sheet or strip of material with very little material waste. Conveniently the sheet or strip can be wound into a roll so that a compact collection of shields is produced. This may also provide an alternative method of dispensing the shielding devices, in which they are removed one-by-one from the roll rather than being detached from a pad or booklet. Accordingly the methods outlined above may further comprise winding the sheet of material into a roll and optionally dispensing the shielding devices from the roll.

It is a common feature of the aspects and embodiments of the invention outlined above that the attachment portion comprises one or more elongate members to attach around a limb of a wearer in use. The following features may be provided in combination with any of the embodiments described herein.

The elongate member of the attachment portion that attaches around a limb of a wearer is preferably flexible. The elongate member can therefore adapt to limbs of different sizes and/or shapes. Furthermore, the elongate member is preferably adjustable in its length. In other words, the distance that the elongate member circumscribes around the limb is preferably adjustable. This may be achieved by the elongate member being elastic or extensible, or by the elongate member wrapping around the limb in an adjustable manner. The elongate member may, for example, comprise an elastic cuff, drawstring or adjustable collar. In some embodiments the elongate member may take the form of an inflatable cuff or other tourniquet, thereby providing the additional function of cutting off blood flow to the limb while shielding. Any kind of necklace, bracelet, anklet, etc. may be used for the elongate member. The elongate member may take the form of a snap band, i.e. a bistable spring strip of metal or plastic that can be converted from a straight (tensioned) state to a curved (untensioned) state when snapped onto a limb.

The elongate member may be adjustable by attaching around a limb and fastening to the shield portion in more than one location. In another set of embodiments the attachment portion may comprise more than one elongate member. The overlap of the elongate members may be adjustable in order to adjust the length of the attachment portion. The elongate member(s) may fasten to one another, or to the shield portion, in any suitable manner. For example, at least one elongate member may comprise a fastening panel. The fastening panel may comprise an adhesive or hook-and-loop (e.g. Velcro™) fastener. In another set of embodiments, at least one elongate member may comprise one or more discrete fasteners such as a hook, clip, button, press stud, etc.

In a preferred set of embodiments the attachment portion comprises an elongate member provided with an adhesive panel. The adhesive panel may be located at an end portion of the elongate member, so as to overlap with another elongate member or with the shield portion of the device. An adhesive panel can be a fastening means that is particularly suitable when the shielding device is integrally provided by a sheet of material, especially a lightweight or thin sheet of material such as paper. An adhesive fastening can contribute to the shielding device being cheap to make and suitable for disposal after a single use. In one set of embodiments the adhesive panel may be provided with a removable cover. A user (the wearer or another person) can then remove e.g. peel off the cover when the elongate member has been wrapped around a limb and the shielding device is in position, so as to adhesively fasten the attachment portion. In another set of embodiments the adhesive panel may automatically be exposed when the shielding device is dispensed, in particular when multiple sheets are bound together as a pad of shielding devices.

In various embodiments it is preferable for the elongate member(s) to be adjustable not only to match the length of the attachment portion to the circumference of a particular limb, but also so that the attachment portion grips onto the limb. It is therefore preferable for the elongate member(s) to be tightenable around a limb of a wearer in use. This can ensure that the shielding device is not just loosely attached to the limb, which could allow the shield portion to rotate or otherwise move around, but attached at a particular location with the shield portion maintained in a desired position. By tightening the elongate member(s) around the limb the attachment portion can be fitted to any size or shape of limb. Furthermore, when the attachment portion grips onto a limb it makes it easer for the shield portion to stay in the same position even if the wearer moves around.

The present invention also extends to a method of attaching a shielding device to a patient so as to shield at least part of a limb from the patient's view, the method comprising: wrapping an elongate portion of the device around the limb; fastening the elongate portion so as to attach the device to the limb; and providing a shield portion of the device in a self-standing configuration. Preferably the self-standing configuration is formed at an angle θ from the limb, where 0<θ<90°.

As is described above, such a method may further comprise converting the shield portion from a first, planar, configuration to a second, three-dimensional configuration. The method may comprise bending the shield portion at one or more hinge lines. In some examples, this may be facilitated by the shield portion comprising at least one curved hinge line. Preferably the shield portion is folded along the curved hinge line and positioned with its folded edge following the contour of the limb. The method may comprise flexing the shield portion to convert it from a first, substantially planar configuration to a second, three-dimensional configuration. In some examples, the shield portion may be flexed into a three-dimensional e.g. curved shape by flexing an edge portion, preferably an edge portion having an outer edge that is at least partially divided into independently moveable sections.

Some further embodiments will now be described, which may be taken alone or in combination with any of the aspects and embodiments of the invention described hereinabove.

The Applicant has recognised that when a shielding device is worn to block a patient's view of the site of a medical procedure, the device can conveniently be used to carry one or more other articles that may be used by a practitioner when carrying out the procedure. This can save the medical professional from reaching away during procedure. In a set of embodiments there is provided a shielding device that is provided with at least one adhesive patch for use before and/or after a procedure. The adhesive patch can be removed from the device and adhered to a patient undergoing the procedure. Where a shielding device is used for a medical procedure it will not be a major procedure requiring a general anaesthetic as the patient is conscious. The medical procedure is likely to be a local procedure—carried out with or without local anaesthetic—such as, for example: drawing blood; inserting a cannula e.g. for intravenous (IV) therapy, infusion or dialysis; inserting a catheter; minor surgery; cleaning and/or dressing a wound; suturing, stapling or otherwise closing a wound; cauterisation; cryotherapy; taking a biopsy; laser treatment; electrolysis and/or tattooing (or any other superficial dermatological procedure). This is not an exhaustive list, and non-medical procedures may also be included. A user of the shielding device can conveniently remove an adhesive patch and adhere it to the limb so as to provide local treatment without needing to turn their attention away from the procedure. Furthermore this can prevent the wearer from seeing the adhesive patch being applied behind the shielding device.

This is considered novel and inventive in its own right, and thus when viewed from a further aspect the present invention provides a device for shielding from view at least part of the body of a wearer of the device, the device comprising a self-standing shield portion, a portion to locate over or around a body part of the wearer in use, and at least one adhesive patch for use before and/or after a procedure. Preferably the adhesive patch is removable from the device so as to be adhered to a wearer of the device. It will be appreciated that the shielding device may take any suitable form for locating over or around a body part of the wearer and shielding his/her view. The shielding device could, for example, comprise a rigid board that locates over or around part of the body such as the neck, torso, abdomen, etc. However in many embodiments the shielding device may comprise an attachment portion comprising at least one elongate member to attach around a limb of a wearer in use. The elongate member(s) can be used to wrap closely around the limb and adapt to different sizes and/or types of limbs e.g. arms, wrists, hands, legs, ankles, feet, etc. . . . .

The at least one adhesive patch may be provided anywhere on the shielding device. In embodiments where the shield portion is removably attached to a spine or spare portion of material, such as a front or back cover sheet, the adhesive patch(es) may be carried by the spine or spare portion. The shielding device can then be considered as part of a kit so that a user has both the shielding device and the adhesive patch(es) to hand when performing a procedure. In one set of embodiments, at least one adhesive patch is provided on a surface of the shield portion. This can be particularly convenient as it means that the adhesive patch is available at the site of the procedure. While one or more adhesive patches may be provided on either surface of the shield portion, it is preferable for the patch(es) to be provided on a side that is facing away from the wearer in use, i.e. on the side accessible to the practitioner that is carrying out the procedure. In some examples the shield portion comprises one or more hinge lines enabling it to be converted from a first configuration in which it is substantially planar to a second configuration in which it has a three-dimensional shape. In other examples, the shield portion may be flexed into a curved three-dimensional shape by flexing an edge portion, preferably an edge portion having an outer edge that is at least partially divided into independently moveable sections. Preferably at least one adhesive patch is provided on a side of the three-dimensional shield portion that is facing away from the wearer in use. Where the shield portion comprises a curved hinged line or a curved edge portion and the second configuration has a curved shape, the adhesive patch is preferably provided on a concave side e.g. so as to face towards the site of the procedure.

Any adhesive patch having a therapeutic effect may be provided. The adhesive patch may be chosen from, for example: a patch for topical administration of a pharmaceutical preparation; an antiseptic wipe; a dressing; an absorbent pad; a tape strip; and an information label. Patches for topical administration of a pharmaceutical may include local anaesthetics such as EMLA (eutectic mixture of local anaesthetics), lidocaine, prilocaine, tetracaine, epinephrine, etc. that can be applied before a procedure. Or patches for topical administration of a pharmaceutical may include an anti-bleeding agent, anti-inflammatory agent, antiseptic agent, etc. that can be applied after a procedure. Other types of patch, such as an antiseptic wipe, dressing, absorbent pad, or tape strip (e.g. steri-strip), can be applied to the wearer to help before, during and/or after a procedure e.g. to manage bleeding and close a wound. An information label may not be applied to the wearer but instead used by the person carrying out the procedure e.g. to label a blood sample.

It will be understood that an adhesive patch may be any substantially planar item that can be adhered to a surface. The item may be provided with a physically present adhesive, e.g. comprising a glue layer or the like. The adhesive may be biologically compatible e.g. when the patch is to be adhered to the surface of a limb or body part. Some forms of adhesive patch may rely on other adhesion methods, such as chemical reaction between the patch and a surface that it contacts, or such as frictional adhesion. In any case adhesion may be assisted by pressure, e.g. when the patch is pressed into contact with a surface.

The Applicant has also recognised that a shielding device can beneficially find use during procedures for the insertion of a sub-dermal implant. These are minor procedures carried out under a local anaesthetic (which may be applied using an adhesive patch as described above instead of a conventional anaesthetic injection or spray). Thus in a set of embodiments the shielding device is part of a kit for insertion of a sub-dermal implant.

This is considered novel and inventive in its own right, and thus when viewed from a further aspect the present invention provides a kit for insertion of a sub-dermal implant, comprising: a sub-dermal applicator preloaded with an implant; a shielding device comprising a self-standing shield portion for shielding from view at least part of the body of a wearer of the device; and, optionally, at least one adhesive patch for use before or after the insertion procedure. Preferably the adhesive patch is removable from the device so as to be adhered to a wearer of the device. The adhesive patch(es) may have any of the features described hereinabove. As before, the shielding device may take any suitable form for locating over or around a body part of the wearer and shielding his/her view. For example, the shielding device may locate over the torso or abdomen when inserting an implant in the abdominal area. In another example, the shielding device may locate over or around the arm when inserting an implant in the arm. In at least some embodiments the shielding device may comprise an attachment portion comprising at least one elongate member to attach around a limb of a wearer in use. The sub-dermal implant may provide hormone therapy, brachytherapy i.e. local radiotherapy, and/or drug therapy. Hormone therapy may include, for example, implants for contraception or treatment of the menopause and implants for treatment of cancer e.g. prostate or breast cancer. Contraceptive implants may include Norplant or Nexplanon®.

As well as the shielding function of the device during the insertion procedure, such kits can take advantageous of the capacity of the shield portion, and any spine or spare portion of material removably attached to the shield portion, to carry information. This can be particularly beneficial when inserting a sub-dermal implant as it may be a procedure that is not regularly carried out by medical professionals. A portion of the shielding device can be used to carry information relating to the use of the sub-dermal applicator. In addition, or alternatively, a portion of the shielding device can be used to carry information for the wearer. For example, a spare portion of the device may be removed and given to the patient as a record of when the implant was inserted. Where an adhesive patch is provided, as least one adhesive patch may take the form of an adhesive label that can be affixed to the patient's medical record. These information-bearing portions of the device may be pre-printed and/or provide a writeable surface.

It is mentioned above that graphics and/or text may be printed on at least one surface of the shield portion of a device. These may be used to distract and/or inform a wearer of the device if provided on a surface facing the wearer. Information or instructions may be provided on a surface facing the person carrying out a procedure behind the device. In one set of embodiments, graphics and/or text are printed on both surfaces of the shield portion. This can provide flexibility when attaching the device to a wearer and selecting which surface will face the wearer during use. The Applicant has recognised that the ability of a shielding device to distract and/or inform a wearer may be augmented by the shield portion comprising a display screen. Rather than static graphics and/or text, a screen can provide a changeable and controllable display. This is considered novel and inventive in its own right, and thus when viewed from a yet further aspect the present invention provides a device for shielding from view at least part of the body of a wearer of the device, the device comprising a self-standing shield portion, a portion to locate over or around a body part of the wearer in use, and a display screen. Of course what is meant by a display screen is an electronic visual display. As before, the shielding device may take any suitable form for locating over or around a body part of the wearer and shielding his/her view. In at least some embodiments the shielding device may comprise an attachment portion comprising at least one elongate member to attach around a limb of a wearer in use.

The shield portion may be integrally provided with a display screen, for example the shield portion itself could be a display screen. This may be appropriate if the shielding device is intended to be re-used e.g. cleaned between uses. However it may be preferable for the display screen to be removably attached to a surface of the shield portion. This can allow the display screen to be re-used while the device is discarded after each single use, to maintain hygiene standards. In either case it can be preferable for the display screen to be flexible or shaped to adapt to a three-dimensional configuration of the shield portion. The display screen could, for example, comprise an LED or OLED display.

These and other features and improvements of the present application and the resultant patent will become apparent to one of ordinary skill in the art upon review of the following detailed description when taken in conjunction with the several drawings and the appended claims.

### BRIEF DESCRIPTION OF THE DRAWINGS

Some preferred embodiments of the present invention will now be described, by way of example only, and with reference to the accompanying drawings, in which:

FIG. 1 relates to an embodiment of a shielding device comprising a removable shield portion;

FIG. 2 relates to an embodiment of a shielding device comprising a snap-action attachment portion;

FIG. 3 relates to an embodiment of a shielding device with a tightenable attachment portion;

FIGS. 4a-4f relate to an embodiment of an integrally formed shielding device;

FIGS. 5a and 5b relate to a first embodiment of an integrally formed shielding device;

FIGS. 6a and 6b relate to a second embodiment of an integrally formed shielding device;

FIGS. 7a and 7b relate to a third embodiment of an integrally formed shielding device;

FIGS. 8a and 8b show a fourth embodiment of an integrally formed shielding device;

FIGS. 9a and 9b-9d show a preferred embodiment of an integrally formed shielding device;

FIG. 10 shows a dispenser of shielding devices in the form of a pad;

FIG. 11 shows a dispenser of shielding devices in the form of a packet;

FIGS. 12a-12d shows a first example of a shielding device being dispensed from a spine;

FIG. 13 shows a second example of a shielding device being dispensed from a spine;

FIG. 14 shows a separated stack of shielding devices;

FIG. 15 shows shielding devices formed in a continuous strip of material;

FIGS. 16a-16c show an embodiment of a shielding device packaged with one or more pre-procedure patches;

FIGS. 17a-17c show an embodiment of a shielding device packaged with one or more post-procedure patches;

FIGS. 18a-18c show an embodiment of a shielding device packaged with both pre- and post-procedure patches;

FIG. 19 shows a typical workflow using a shielding device according to any of FIGS. 15-17;

FIG. 20 shows a shielding device packaged with an implant insertion kit; and

FIGS. 21a-21c show some embodiments of a shielding device comprising different display screens.

FIG. 22 shows an embodiment of an integrally formed shielding device with the shield portion having a softened outer edge;

FIG. 23 shows an alternative embodiment of an integrally formed shielding device with the shield portion having a cut edge portion;

FIGS. 24a and 24b show shielding devices formed from a strip of material so as to be dispensed from a roll;

FIGS. 25a and 25b show an alternative embodiment of shielding devices formed from a strip of material for dispensing from a roll;

FIG. 26 shows an embodiment of an integrally formed shielding device without any hinge lines; and

FIGS. 27a, 27b, 27c, and 27d show an alternative embodiment of an integrally formed shielding device without any hinge lines.

### DETAILED DESCRIPTION

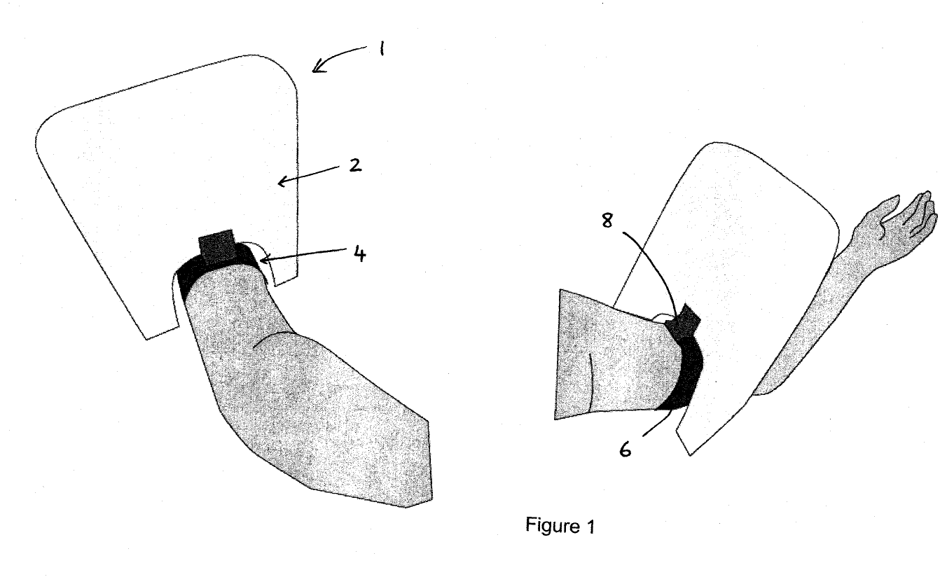

There is seen in FIG. 1 an embodiment of a shielding device 1 comprising a shield portion 2 that is removable from an attachment portion 4. In this example the shield portion 2 is a rigid piece of material such as cardboard, plastic or a laminate thereof. The shield portion 2 is shown as a planar piece of material but it may instead have a three-dimensional shape. The attachment portion 4 takes the form of an elongate member 6 that wraps around a limb such as an arm. The elongate member 6 may be a bracelet that grips onto the arm. The attachment portion 4 also includes a holder 8 fixed onto the elongate bracelet 6. A chosen shield portion 2 can be slid in and out of the holder 8. The shield portion 2 may therefore be removed and disposed of while the attachment portion 4 is re-used. Different shield portions 2 may be used with the attachment portion 4 for different purposes.

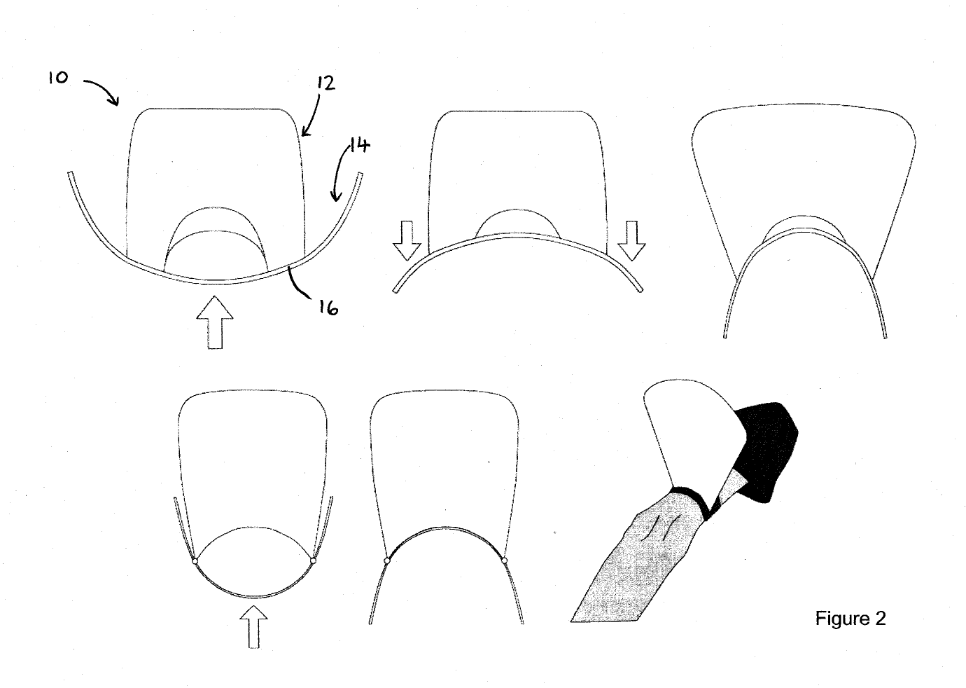

FIG. 2 shows another embodiment of a shielding device 10 comprising a shield portion 12 attached to an adjustable attachment portion 14. The attachment portion 14 is in the form of an elongate snap band 16. It can be seen that the snap band 16 can be pushed to reverse its curvature and wrap around a limb. The shielding device 10 readily adapts to limbs of different shapes and sizes. In this example the shield portion 12 may be permanently connected to the attachment portion 14 or it can be removable.

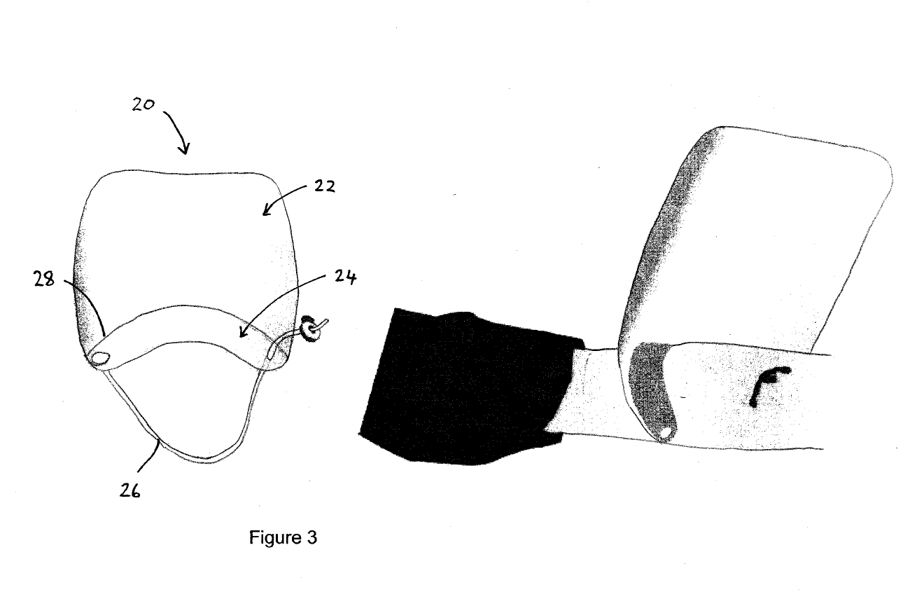

In FIG. 3 there is shown another embodiment of a shielding device 20. The shield portion 22 may be formed from a flexible but stiff material such as card or sheet plastic. The shield portion 22 in connected to an attachment portion 24 of the same material by a hinge line 28. The hinge line 28 is curved so that the shield portion is bent into a three-dimensional curved shape. The attachment portion 24 is provided with an elongate drawstring 26 that can be tightened around a limb, as shown. The drawstring 26 is adjustable to account for different limb sizes.

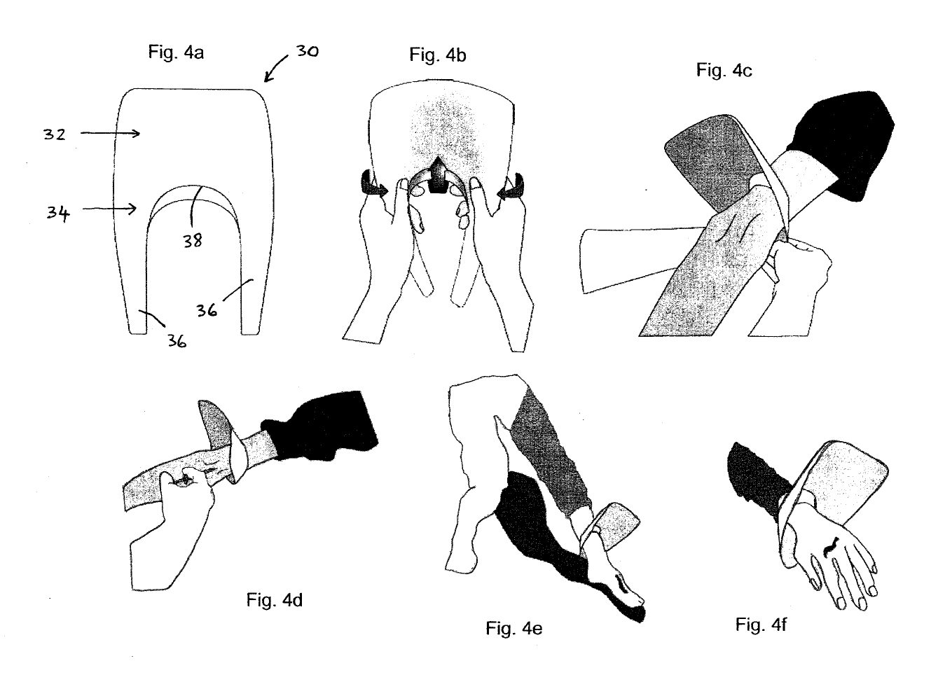

FIGS. 4a-4f show an integrally formed shielding device 30, e.g. made from a piece of plastic, card or paper material. The device 30 has a shield portion 32 and an attachment portion 34 comprising a pair of elongate legs 36. The shielding device 30 may have an initial configuration that is planar (FIG. 4a), but can be converted to a three-dimensional shape by folding along a hinge line 38 (FIG. 4b). The three-dimensional shield portion 32 can be attached to a limb by wrapping the legs 36 around the limb and fastening them together (FIG. 4c). Any suitable fastening means may be used, for example adhesive fastening or hook-and-loop (e.g. Velcro™) fastening. The shield portion 32 has an edge portion defined by the hinge line 38 that is folded back to form a base for the upstanding remainder of the shield portion 32. It can be seen that the curved hinge line 38 formed a folded edge that is also curved so that it can be easily wrapped around a limb. FIG. 4d shows the shielding device 30 attached around an arm to shield an injection from view. FIG. 4e shows the shielding device 30 attached around an ankle to shield a wounded foot from view. FIG. 4f shows the shielding device 30 attached around a wrist to shield a wounded hand from view. The shielding device 30 may also have a protective function, especially for wounds or other injuries.

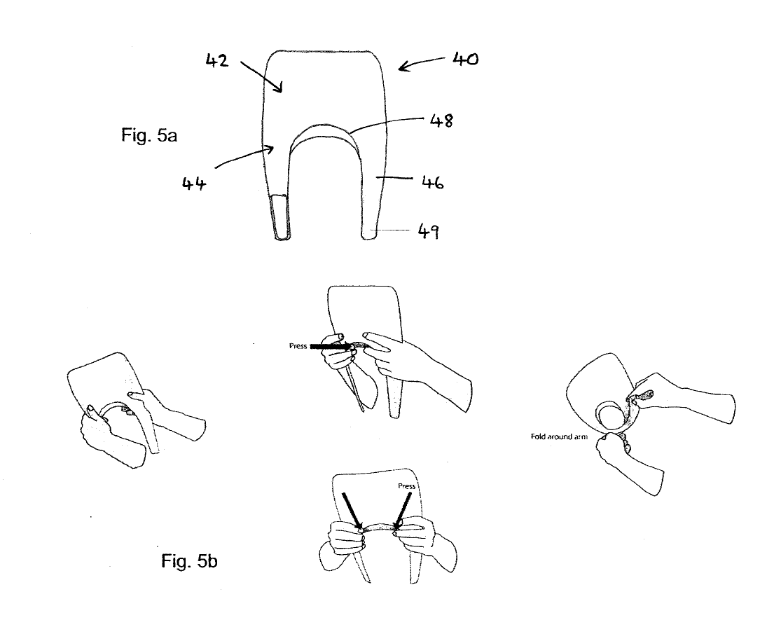

FIGS. 5a and 5b illustrate an integrally formed shielding device 40 similar to that seen in FIGS. 4a-4f. The device 40 has a single hinge line 48 that is curved to enable the shield portion 42 to be bent from an initially planar configuration (FIG. 5a) to a curved three-dimensional shape (FIG. 5b). The attachment portion 44 of the device 40 is made up of two legs 46 that are each provided with an adhesive panel 49. Once the legs 46 have been wrapped around a limb then they can be attached to one another, or to the shield portion 42, using the adhesive panels 49. Although not shown, the adhesive panels 49 may optionally be provided with removable covers. In other versions of the device 40, only one of the legs 46 may be provided with an adhesive panel. Furthermore it will be appreciated that it is not necessary for the attachment portion 44 to comprise two legs, and instead a single leg could wrap around the limb to fasten to the shield portion 42 on the other side.

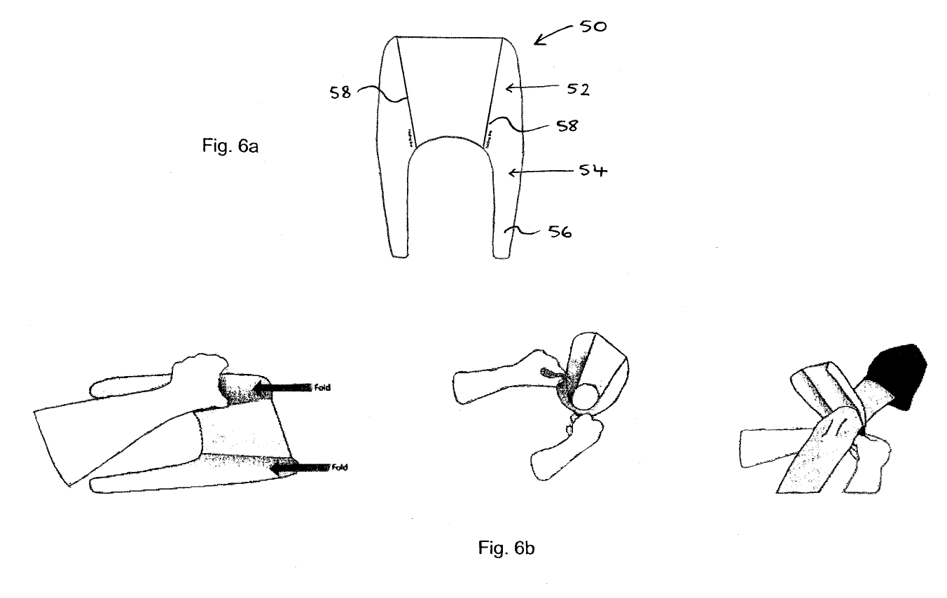

FIGS. 6a and 6b illustrate an integrally formed shielding device 50 similar to that seen in FIGS. 4a-4f and FIGS. 5a-5b, except that this device 50 has a pair of hinge lines 58. The two straight hinge lines 58 enable the shield portion 52 to be bent from an initially planar configuration (FIG. 6a) to an angular three-dimensional shape (FIG. 6b). As before, the attachment portion 54 of the device 50 is made up of two legs 56 that can wrap around a limb and be fastened to one another. The legs 56 may be fastened using an adhesive panel or any other fastener such as Velcro™ or even press studs.

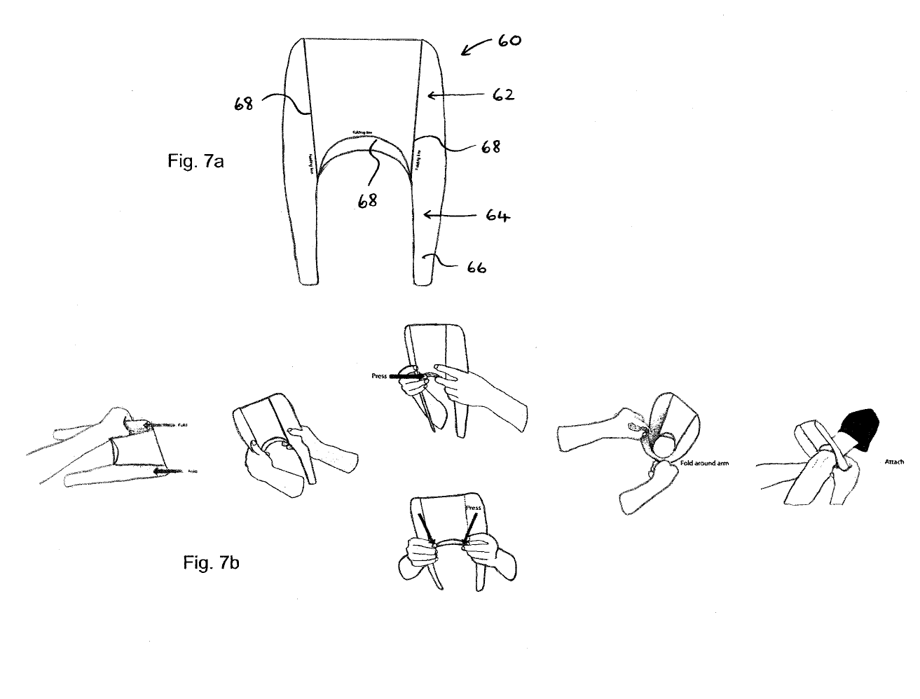

FIGS. 7a and 7b illustrate an integrally formed shielding device 60 that combines the features of FIGS. 5a-5b and FIGS. 6a-6b. This device 60 is provided with multiple hinge lines 68 that enable the shield portion 62 to be bent from an initially planar configuration (FIG. 7a) to a complicated three-dimensional shape (FIG. 7b). As before, the attachment portion 64 of the device 60 is made up of two legs 66 that can wrap around a limb and be fastened to one another.

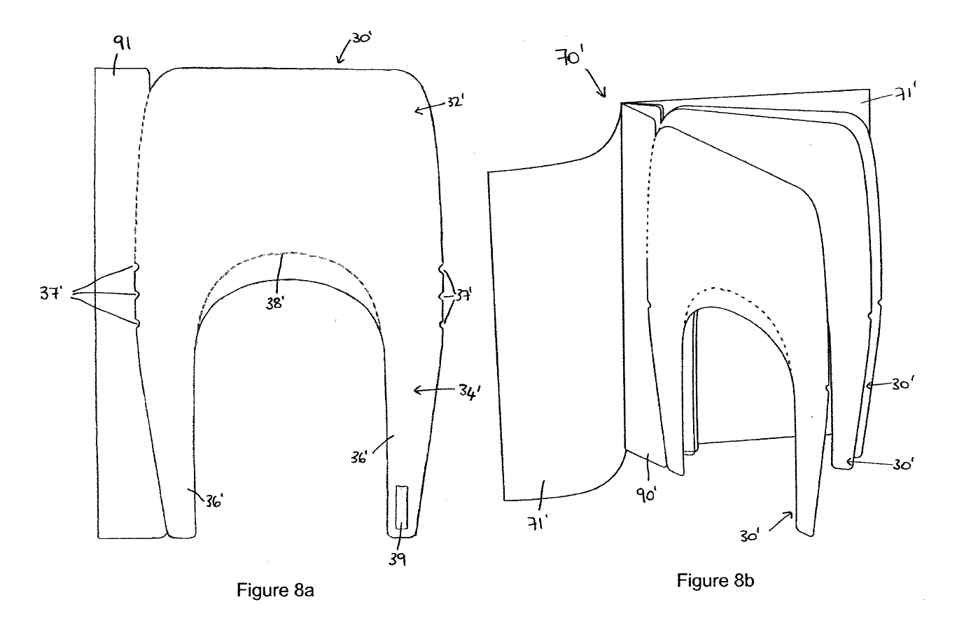

FIGS. 8a and 8b show another embodiment of an integrally formed shielding device 30′ that is similar to that seen in FIGS. 4a-4f and FIGS. 5a-5b. The device 30′ is made from a single sheet of plastic, card or paper material. As before, the device 30′ has a shield portion 32′ and an attachment portion 34′ comprising a pair of elongate legs 36′. The initial configuration of the shielding device 30′ is planar, but the device 30′ can be converted to a three-dimensional shape by folding along a curved hinge line 38′ and then attached to a limb in the same manner as is shown in FIGS. 4b-4f. The legs 36′ can be wrapped around the limb and fastened together using an adhesive panel 39 (which may be provided with a removable cover to expose the adhesive during use).

The dashed lines in FIGS. 8a and 8b represent perforations while the solid lines represent a cut edge. It can be seen that the curved hinge line 38′ is perforated, which helps to ensure that the device 30′ can easily be folded in either direction into its 3D configuration. The device 30′ may have different graphics and/or text displayed on the back and front, so that folding it one way or the other determines which side is displayed to a wearer when the device 30′ is attached. The device 30′ is integrally formed with a spine 91 that extends down one side. So as to facilitate detachment of the device 30′ from the spine 91, this side of the device 30′ is cut along part of its length (solid line) and perforated along the rest of its length (dashed line). It may also be seen that three notches 37′ are cut into each side of the device 30, located approximately between the shield portion 32′ and the attachment portion 34′. These notches 37′ make it easier to remove the device 30′ from a limb after use. Instead of trying to pull apart the legs 36′ where they are attached at the adhesive panel 39, the notches 37′ can be used to tear the shield portion 32′ away from one of the legs 36′ and release the device. This means that a relatively strong adhesive can be used for the panel 39. A further advantage of tearing off the device 30′ is that it is destroyed and can not be re-used, which is more hygienic.

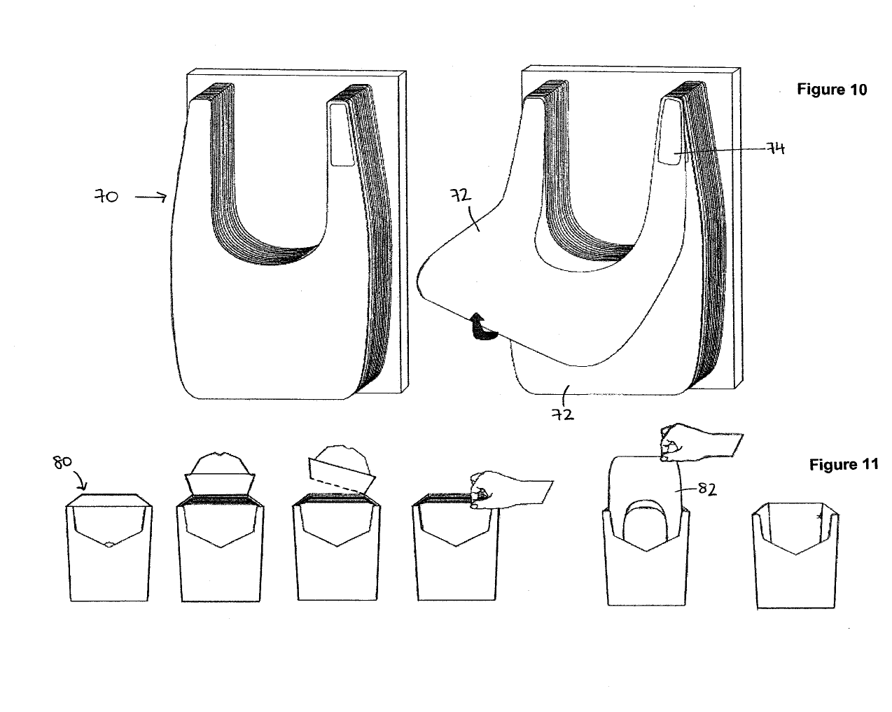

FIG. 8b shows an example of a booklet-type dispenser 70′ for the shielding devices 30′. Each device 30′ takes the form of a sheet in the booklet 70′, with the sheets being bound together by their spines 91. The booklet 70′ has front and back covers 71′ to protect the shields 30′ and help keep them clean before use. The devices 30′ can be dispensed one-by-one by tearing them away from their spines 91. It is advantageous that each device 30′ is cut along the spine 91 next to the leg 36′ so that a user does not try to pull the leg 36′ to release the device 30′, which could accidentally result in a leg 36′ being torn off. Instead a user pulls the shield portion 32′ away from the spine 91 to release the device 30′ by tearing along the perforated line, so that the device 30′ stays intact. Some alternative dispenser designs will be described in relation to FIGS. 10-14.

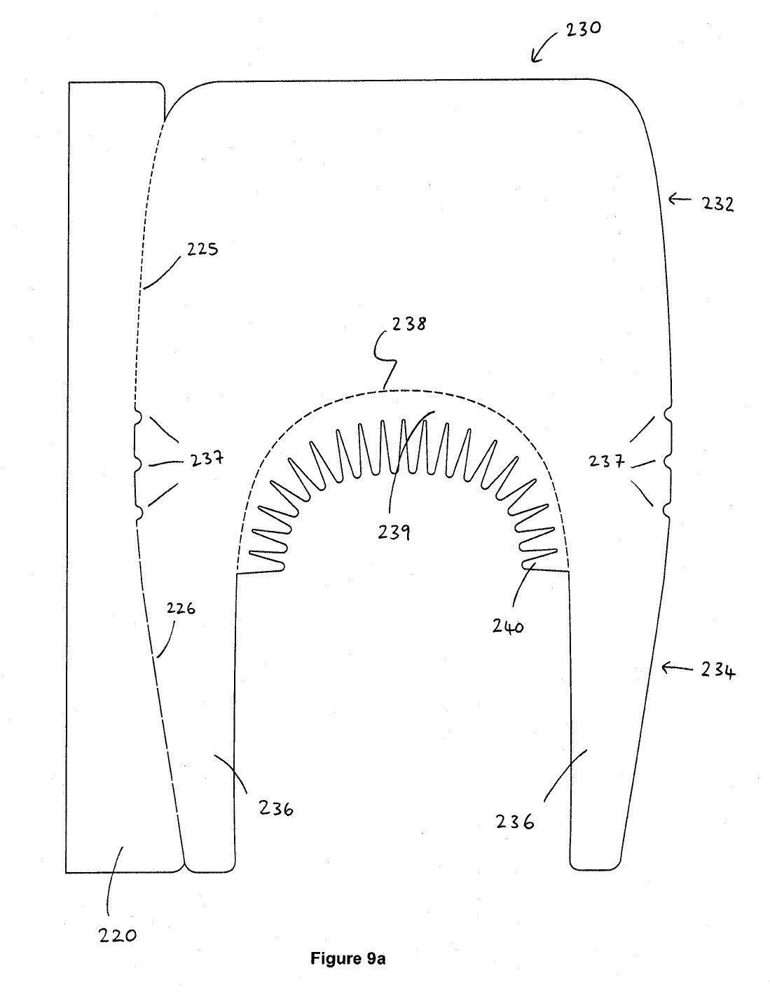

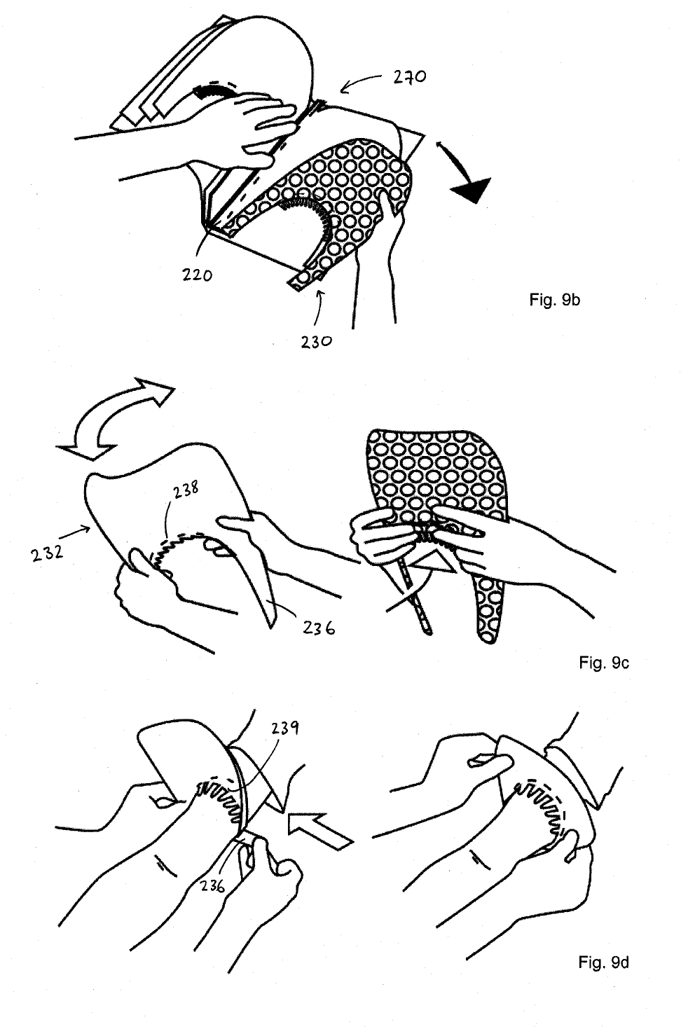

FIGS. 9a and 9b-9d show a preferred embodiment of an integrally formed shielding device 230 that is an improved version of the device 30′ seen in FIGS. 8a and 8b. Again the device 230 is made from a single sheet of relatively rigid sheet material such as plastic, card or paper. A thin sheet e.g. 1 mm paper or card is preferred. As before, the device 230 has a shield portion 232 and an attachment portion 234 comprising a pair of elongate legs 236. The device 230 is integrally formed with a spine 220 that extends down one side. The device 230 is connected to the spine 220 by a perforated line 225. It can be seen that three notches 237 are cut into each side of the device 230, located approximately between the shield portion 232 and the attachment portion 234. These notches 237 make it easier to remove the device 230 from a limb after use, as they define a starting point for tearing off the device 230 after use. The leg 236 on the same side as the spine 220 is freed by a cut line 226 extending along the edge of the device 230 to the notches 237. It can be seen from FIG. 9b that the shielding device 230 may take the form of a sheet that is bound by its spine 220 into a booklet dispenser 270, to be dispensed in the same manner as is described above with respect to FIG. 8b.

The initial configuration of the shielding device 230 is planar, as is seen in FIG. 9a, but the device 230 can be converted to a three-dimensional shape by folding along a curved hinge line 238 (FIG. 9c) and then attached to a limb in the manner shown in FIG. 9d. The legs 236 can be wrapped around the limb and fastened together using an adhesive panel (not shown). The shielding device 230 has an edge portion 239 defined by the hinge line 238 which forms a folded edge of the shield portion 232 when it is converted into its 3D configuration. The edge portion 239 has a curved outer edge opposite the hinge line 238. It can be seen that this outer edge is divided into a number of spaced sections 240, similar to teeth, which are connected by a small internal curve, e.g. of 0.5 mm radius. This allows the sections 240 to move relative to one another, changing the angle between adjacent sections as the edge portion 239 is flexed. As can be seen from FIG. 8b, the spaced sections 240 spread around the limb when the shielding device 230 is attached, so that the edge portion 239 more easily forms a curved base conforming to the shape of the underlying limb and any garments (or other foreign body) changing the geometry of the underlying limb. By separating the edge portion 239 into a number of sections 240, there is increased stability, preventing the shield portion 232 from tipping forwards or backwards towards the limb. In addition, the device 230 can both be attached more tightly around the limb, and moved (e.g. turned) more easily once attached to the limb. This is because splitting the edge portion 239 into sections 240 allows the length of the outer edge to change, so that the edge of the sheet material is not likely to cut into the skin while the device 230 is being tightened or moved.

Turning now to FIG. 10, there is seen an example of a dispenser 70 in the form of a pad. The dispenser 70 is made up of a stack of shielding devices 72 that are bound together by adhesive panels 74, which may be on their upper and/or lower surface. The shielding devices 72 are each made from a single piece of material, such as a sheet of paper, and may take any of the forms described above with respect to FIGS. 4-9. The shielding devices 72 can be peeled off from the pad one-by-one. If the devices 72 have an adhesive panel 74 on their upper surfaces then the uppermost device 72 in the pad may be provided with a protective cover (not shown) for the adhesive panel 72. However adhesive panels 74 on the devices 72 behind will automatically be protected by the upper devices until they come to be exposed at the top of the pad.

FIG. 11 shows another example of a dispenser 80, this time in the form of a packet. Sheet-like shielding devices 82 (similar to those described above with respect to FIGS. 4-7) are stacked and held in the packet. The shielding devices 82 may be like the ones seen in FIGS. 8 and 9, but without a spine. An advantage of this dispensing arrangement is that the packet can be closed to protect the shielding devices during transport and storage. Only once the packet has been opened are the devices exposed, which can help to meet clinical standards of hygiene. The shielding devices 82 can be removed from the packet one-by-one. The devices may not be attached to one another so that a user is free to select any device rather than only the front one in the stack.

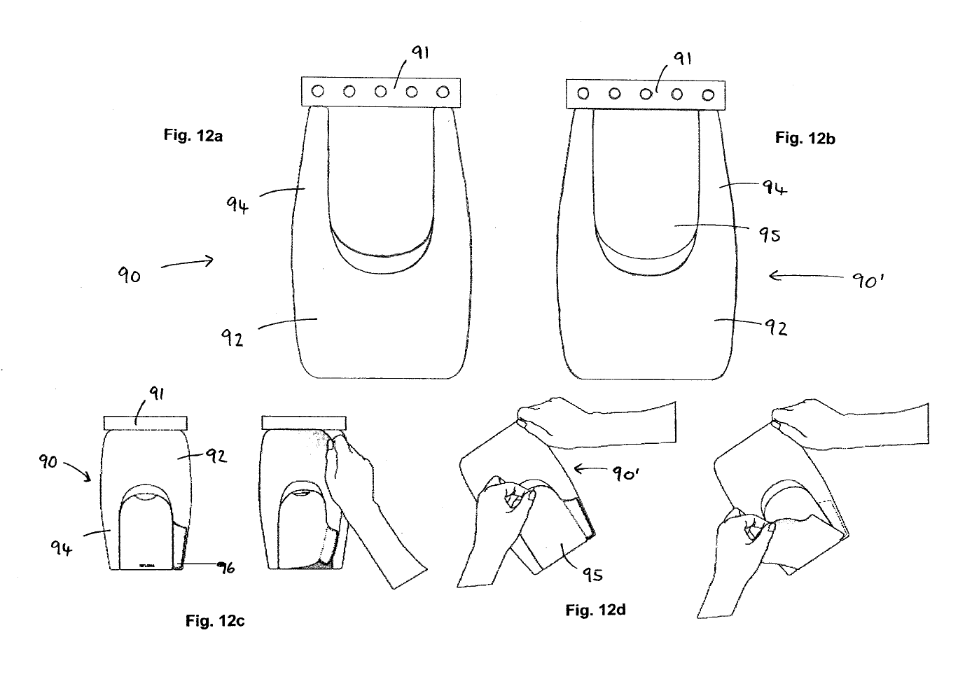

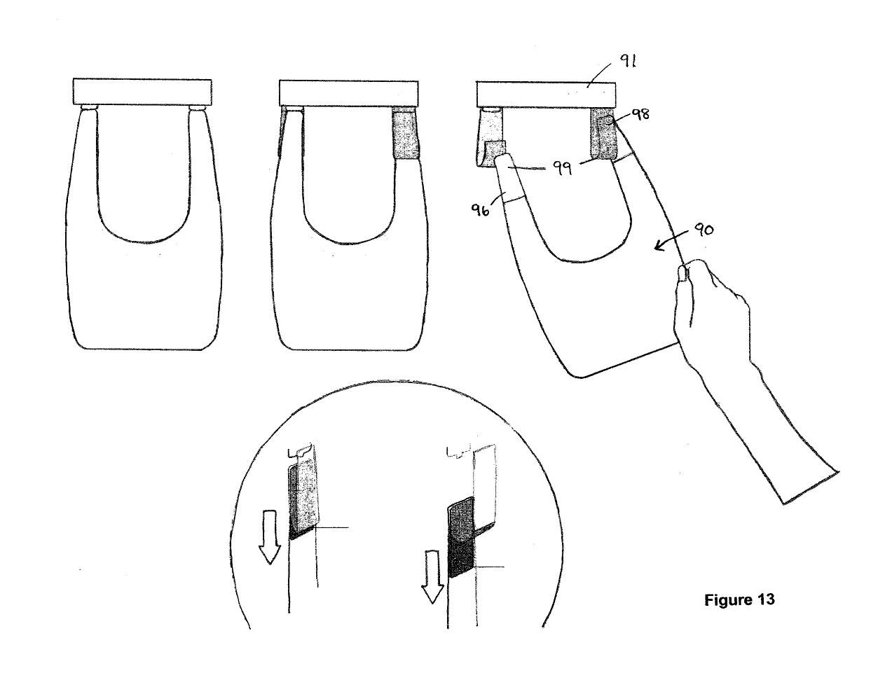

In the examples seen in FIGS. 12 and 13, a shielding device 90, 90′ is dispensed from a spine 91. The spine 91 may be used to hang one or more devices 90, 90′. The spine 91 may be used to bind multiple devices 90, 90′ together in a pad. As is seen in FIG. 12a, the device 90 may already be cut out from a sheet. Or, as is seen in FIG. 12b, the device 90′ and the spine 91 may be formed as a continuous sheet of material. The inner boundary of the device 90′ is perforated to allow a portion 95 of spare material to be removed. In FIGS. 12a and 12b the spine 91 is shown connected to the attachment portion 94 of the device 90, 90′. However the spine 91 may instead be connected to the shield portion 92, as is shown in FIG. 12c. Of course the spine 91 may be connected to any part of the device 90, 90′, for example along a side edge instead of upper or lower edges. In FIG. 12c there are shown the steps for dispensing a device 90, 90′ by removing it from the spine 91, for example by tearing along a perforated line. This may include peeling off the cover of an adhesive panel 96. In FIG. 12d there are shown the optional steps for removing the portion 95 of spare material from a device 90′. Although this spare portion 95 does not form part of the shielding device 90′ in use, it may not be discarded as waste but could serve a purpose as an information carrier, for example being printed with instructions or the like. If the shielding device 90′ is designed for use with infants then the spare portion 95 could take the form of a certificate e.g. congratulating a patient for having had an injection. The shielding device 90′, or at least the spare portion 95, may therefore be provided with a writeable surface.

FIG. 13 illustrates how shielding devices 90 that carry an adhesive panel 96 may be dispensed from a spine 91. The adhesive panel 96 is provided with a cover 98 that is connected to the spine 91. When a device 90 is removed from the spine 91, it is also pulled away from the cover 98 which remains with the spine 91. The glue 99 on the adhesive panel 96 is therefore exposed ready for use as the device 90 is dispensed, and without any need to separately remove or discard the cover 98.

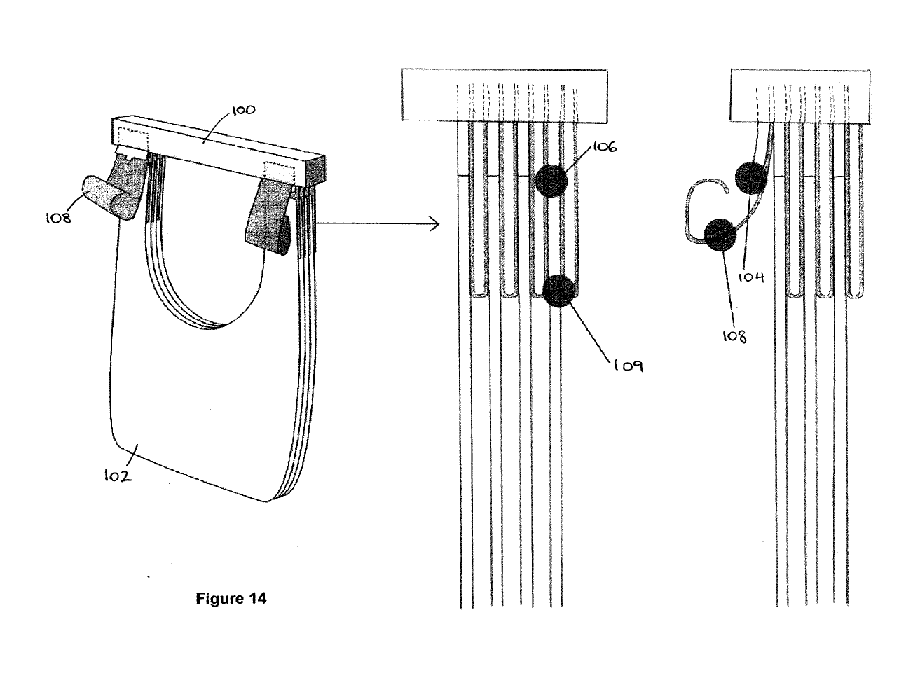

In FIG. 14 there is shown a related example with a stack of separate shielding devices 102 connected to a spine 100. Each device 102 hangs independently from the spine 100 and a user can therefore choose which device 102 to remove rather than having to take the one at the front of the stack. Each device 102 is removably connected at a perforation 106 to one or more stubs 104 (e.g. at the end of the legs) that is/are bound to the spine 100. When a device 102 is torn from the spine 100, the stubs 104 are left behind. The cover(s) 108 also remain behind with the spine 100 so that adhesive 109 on a surface of the device 102 is exposed.

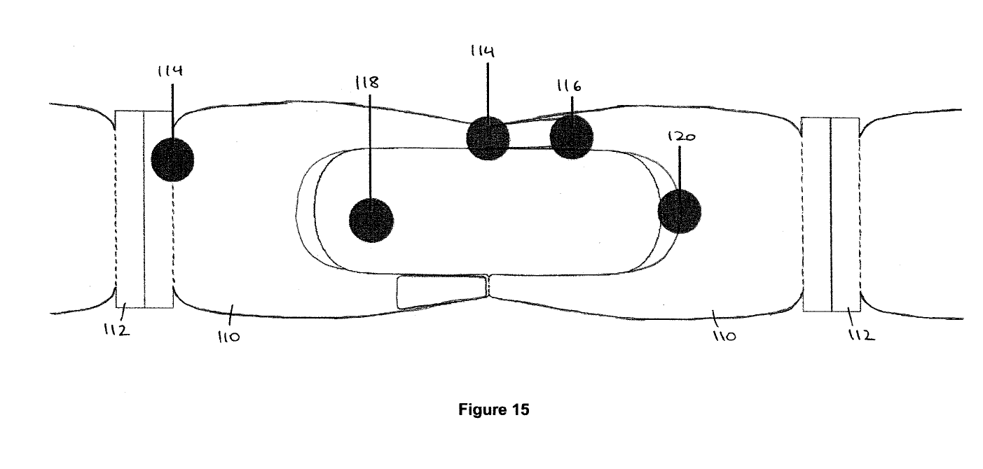

Finally, it can be understood with reference to FIG. 15 that shielding devices 110 formed from a single piece of material, such as paper, can be manufactured in a continuous run. In this example the devices 110 are formed end-to-end with a spine 112 between adjacent pairs of devices 110. The spine 112 may be severed or folded. Perforation lines 114 are formed between the devices so that they can be separated from one another. Also perforation lines 114 are formed between each device 110 and the spine 112 to enable the devices 112 to be dispensed for use. An adhesive panel 116 (with or without a protective cover) is applied to each device 110. A spare portion 118 of material inside each pair of devices 110 may be cut away during manufacture or left attached with a perforated line to be dispensed together with the devices 110 (as discussed above). One or more hinge i.e. fold lines 120 are scored or otherwise formed e.g. perforated in each device 110 to enable it to be converted from a planar configuration to a three-dimensional shape in use.

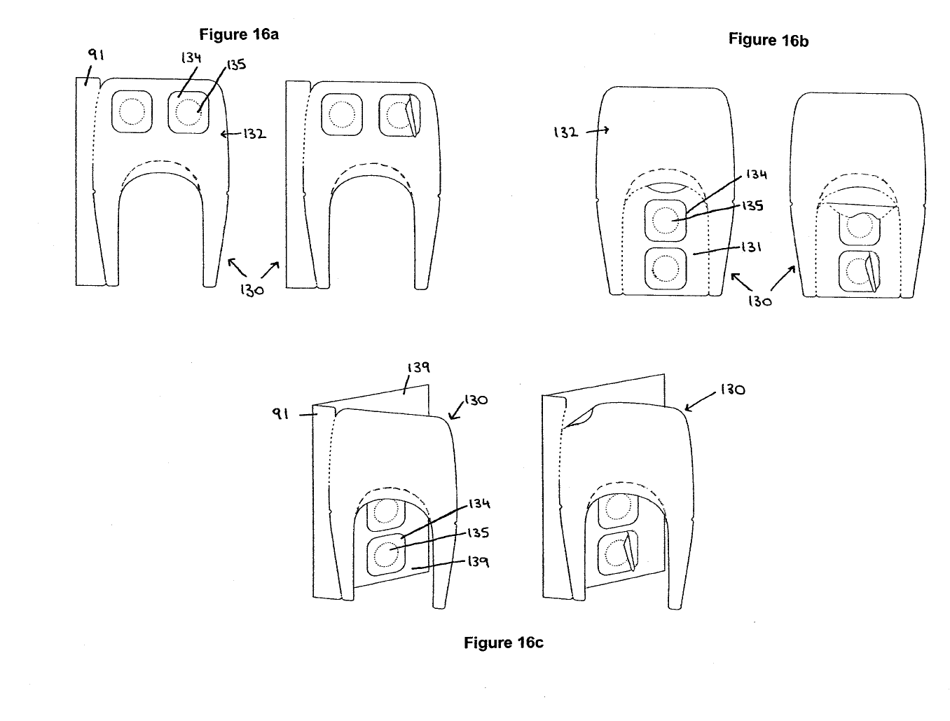

FIGS. 16 to 21 show some further embodiments of an integrally formed shielding device which is similar in its form to the embodiment seen in FIGS. 8a and 8b, e.g. with the shield portion having a curved hinge line enabling it to be converted from a planar configuration to a three-dimensional configuration. Although not shown as having an edge portion with separated sections along its outer edge, the shield portion could combine this feature with the curved hinge line in the same manner as is illustrated in FIG. 9a.

FIGS. 16a-16c are some embodiments of a shielding device 130 packaged with one or more pre-procedure patches 134, e.g. patches for the topical administration of a pharmaceutical preparation 135 such as a local anaesthetic e.g. EMLA. As can be seen from FIG. 16a, the patches 134 may carried by the shield portion 132 of the device. In this embodiment the device 130 is removably attached to a vertical spine 91 that can allow a number of devices 130 to be bound together e.g. in a pad or booklet. Alternatively, as can be seen in FIG. 16b, the patches 134 may be held on a spare portion of material 131 which will can be removed before the device 130 is used. In FIG. 16c, the patches 134 are instead carried by a back cover 139 connected to the device 130 by means of a vertical spine 91. In the embodiments of both FIGS. 16b and 16c, the patches 134 are not attached to the device 130 itself and therefore all packaging can be disposed of ahead of use of the device 130.

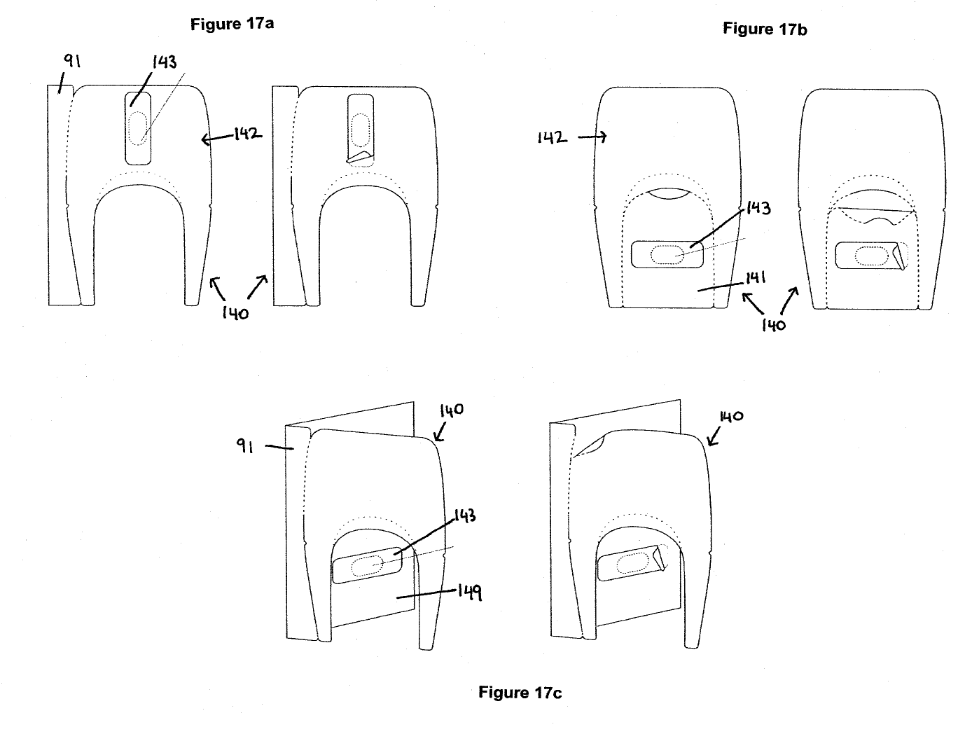

FIGS. 17a-17c are some embodiments of a shielding device 140 packaged with one or more post-procedure patches 143, for example in the form of an adhesive dressing. The patches may also contain some form of pharmaceutical, but typically they are pressure dressings. In a similar manner to FIGS. 16a-16c, these patches 143 may be carried by the shield portion 142, by a removable spare portion 141, or by a back cover 149 of the device. In FIGS. 17a and 17c the device 140 is removably attached to a spine 91 so as to be dispensed from a pad or booklet.

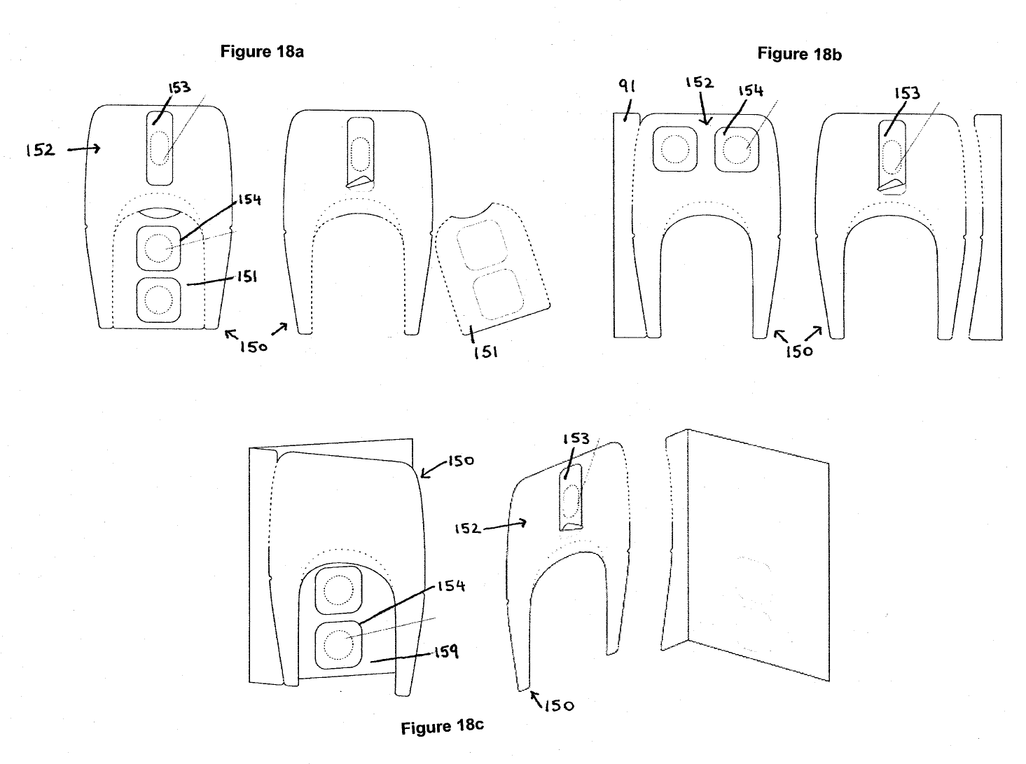

Of course, both kinds of patch could be provided together on the same shielding device. FIGS. 18a-18c illustrate some embodiments of such a combination. The patch 154 and dressing 153 can be carried by the device 150 in a number of different ways. In all three embodiments, the post-procedure dressing 153 is carried by the shield portion 152, such that it is easily accessible at the site of the procedure. FIG. 18a shows an embodiment wherein the pre-procedure patches 154 are carried by a removable portion 151 of the device 150, so that the associated packaging can be disposed of before the device 150 is used. In FIG. 18b, the pre-procedure patches 154 are instead carried on one surface of the shield portion 152 while a post-procedure dressing 153 is carried on the opposite side of the device 150. The pre-procedure patches 154 can be removed prior to a procedure so as not to obstruct any design on the side of the device 150 that faces the wearer during use. The post-procedure dressing 153 is then facing a person carrying out the procedure so that it can easily be applied e.g. after an injection. A number of the devices 150 could be bound together in a pad or booklet using the spine 91. FIG. 18c shows another embodiment, in which the pre-procedure patches 154 are carried by a removable back cover 159 of the device 150, while a post-procedure dressing 153 is carried on a back surface of the shield portion 152.

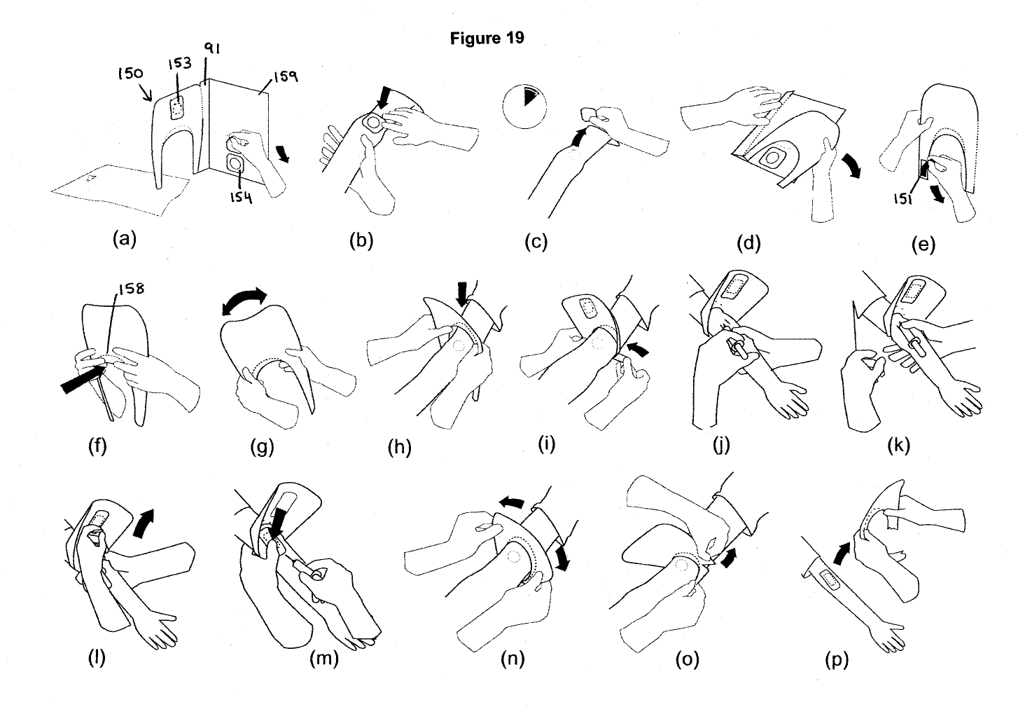

FIG. 19 shows a typical workflow using a shielding device 150 that is packaged with patches 153, 154 for use before and after an injection or other medical procedure. The shielding device 150 (as seen in FIG. 19a) is the same as that described above in relation to FIG. 18c, where the pre-procedure patches 154 are carried by the back cover 159 and the post-procedure dressing 153 is carried on a rear surface of the device 150. First the pre-procedure patches 154 are removed from the back cover 159, and affixed to a limb of the patient for a period of time e.g. to allow a local anaesthetic to take effect (FIGS. 19a and 19b). Once the patch 154 has been removed (FIG. 19c), the shielding device 150 is detached from the spine 91 of the cover 159 ready for use (FIG. 19d). A cover on the adhesive panel 151 is removed (FIG. 19e) and the device 150 is bent along the curved hinge line 158 so as to convert the shield portion from a planar configuration to a curved shape (FIGS. 19f and 19g) before the device 150 is attached to a limb of the patient (FIG. 19h). The device 150 is fastened around the limb using the adhesive panel 151 (FIG. 19i). The procedure, such as injection, is then able to be carried out away from the view of the patient (FIG. 19j). Once the procedure is finished, the post-procedure dressing 153 is readily available on the back surface of the device 150 and can be removed using one hand e.g. while the other hand holds the needle (FIGS. 19k and 19l). The dressing 153 is peeled off from the back of the device 150 and affixed to the patient's limb (FIG. 19m). The device 150 can be rotated around the limb (FIG. 19n) and then torn off (e.g. at one or more weak points as described in relation to FIG. 8 or 9) in order to be removed from the patient and disposed of (FIGS. 190 and 19p).

FIG. 20 is one example of a shielding device 170 packaged as part of a sub-dermal implant insertion kit. Similar to FIG. 17a, a post-procedure dressing 173 is carried by the shield itself for easy access. In this embodiment, the shield 170 is bound with two other sheets, one sheet 179a which holds the other items for the procedure, and the other sheet 179b which is printed with instructions for carrying out the insertion procedure. The items carried by the middle sheet 179a of the booklet are a sub-dermal applicator 172 (pre-loaded with an implant), an ID card 174 for the patient to keep (to show that they have had the implant), information 176 about production and lot numbers, and adhesive labels 178 that may be affixed to a patient's medical record. By packaging all the relevant components and information together in such a kit, the procedure can be carried out much more smoothly, and both doctor and patient can be assured that they have all the necessary information.

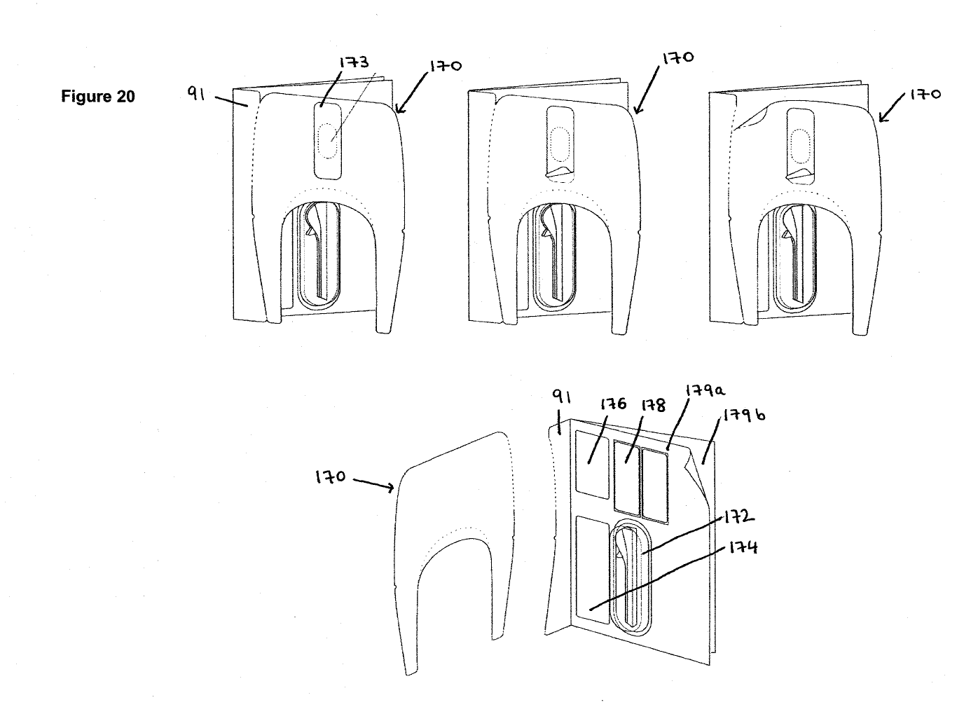

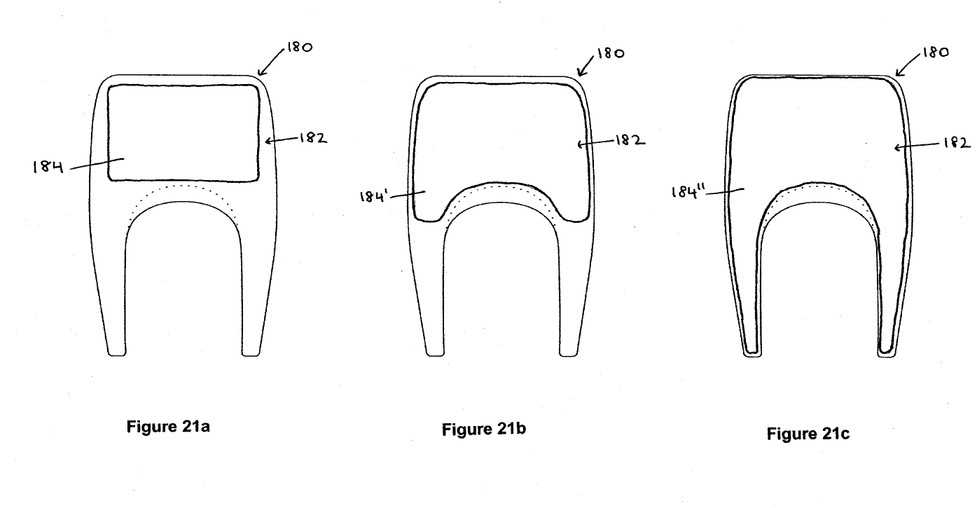

FIGS. 21a-21c show some embodiments of a shielding device 180 comprising different display screens 184, 184′, 184″. This allows for moving images to be shown to the patient in order to distract them from the procedure taking place. The display screen may be integrated with the shielding device 180, or removably attached, e.g. enabling it to be reused for a number of procedures. In one set of examples, a LED or oLED screen 184, 184′ is affixed to the shield portion 182 of the device 180, covering varying amounts of the surface, as can be seen by comparing FIGS. 21a and 21b. Alternatively, in the embodiment of FIG. 21c, the shield 180 may be formed entirely from a LED or oLED display screen 184″. The display screen 184, 184′, 184″ may be a flexible screen so as to be able to conform with the shape of the device 180 as it is wrapped around a limb of a patient.

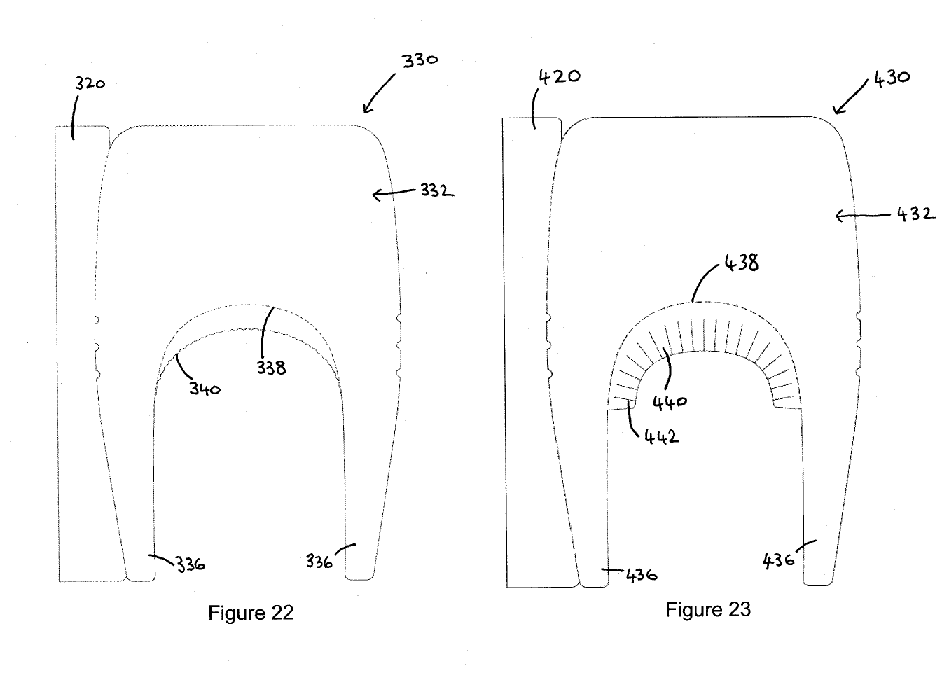

FIG. 22 shows an embodiment of an integrally formed shielding device 330 comprising a pair of elongate legs 336 and a shield portion 332 in which the outer edge 340 is corrugated, in order to soften the edge which is in contact with a wearer's skin. When the edge portion (as defined by the hinge line 338) is folded in order to convert the shielding device 330 to a 3D configuration, the corrugation of the edge 340 provides a softer surface in contact with the skin. This reduces the likelihood of the shielding device 330 cutting a wearer while in use, for example when the shielding device 330 is twisted around a limb to adjust the position of the shield portion 332. The device 330 is shown attached to a spine 320, e.g. formed as a sheet in a pad or booklet, but the device 330 may be dispensed by any other suitable means.

FIG. 23 shows an alternative embodiment in which an integrally formed shield 430 has a shield portion 432 with a softened outer edge portion 440 between the elongate attachment legs 436. In this embodiment, the edge portion 440 has a number of cuts 442, such that it is divided into a number of sections which are able to move relative to one another, changing the angle between adjacent sections as the edge portion 440 is flexed. This allows the edge portion 440 to fit more easily to a limb, and as previously discussed allows the shielding device 430 to be attached more tightly to a limb as the fringed outer edge portion 440 is less likely to cut into the skin. The device 430 is shown attached to a spine 420, e.g. formed as a sheet in a pad or booklet, but the device 430 may be dispensed by any other suitable means.

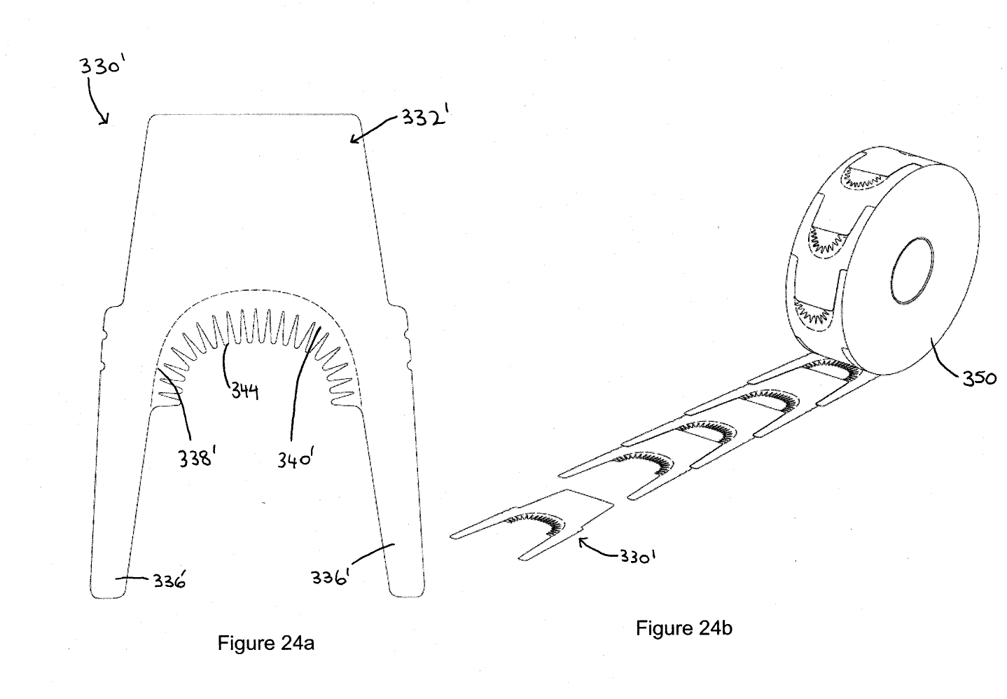

FIG. 24a shows an alternative design of an integrally formed shielding device 330′, which is shaped such that it can be tessellated end-to-end in a strip and dispensed from a roll 350 as seen in FIG. 24b. In this embodiment the shield portion 332′ comprises an edge portion 340′ (defined by a hinge line 338′) that is separated into a plurality of spaced apart teeth 344. The shield portion 332′ is tapered along its length, such that the shield portion 332′ can fit between the elongate legs 336′ of an adjacent shield 330′. This allows a plurality of shielding devices 330′ to be formed from a continuous piece of material, which can then be wound to form a roll 350. This provides an alternative method of dispensing shields, in which it is not necessary to provide an additional spine to detach the shield from.

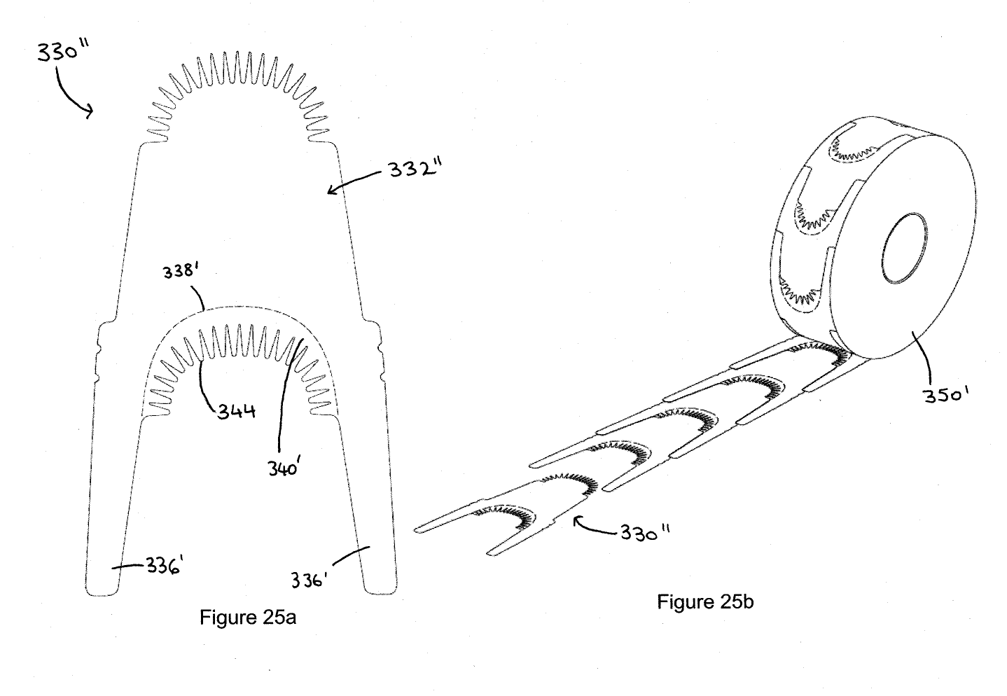

FIGS. 25a and 25b show an alternative embodiment in which shielding devices 330″ are dispensed from a roll 350′. In these embodiments, the shield portion 332″ of each device 330″ is shaped such that it fits exactly within the legs 336′ of an adjacent shield 330″, and there is no wasted material. The outer edge of one shield portion 332″ matches the toothed outer edge 344 of the edge portion 340′ of an adjacent shield 330″, e.g. they are formed with meshing teeth. This embodiment minimises the amount of manufacturing that is needed in forming the shielding devices, as there is no waste material that must be removed, either in the formation of the roll 350′ or in the removal of an individual shield 330″.

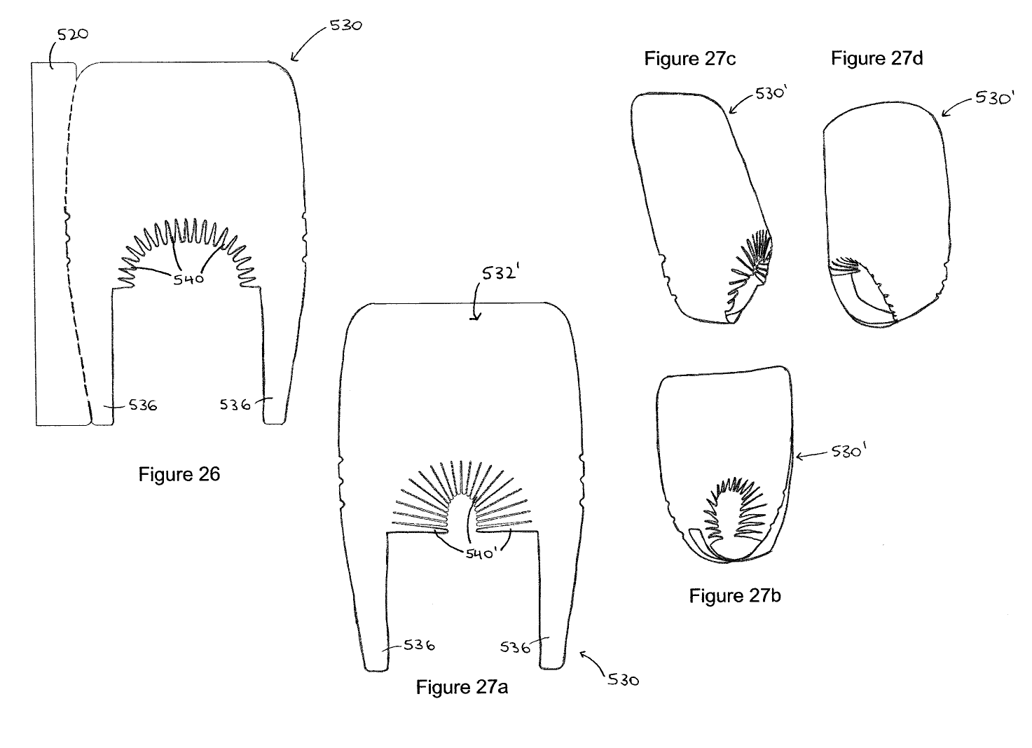

FIG. 26 illustrates an alternative embodiment of an integrally formed shielding device 530. In this embodiment, there are no hinge lines for converting the shield portion 532. Instead, the shield 530 is formed from a flexible material which gives the shield portion 532 a three-dimensional shape due to the way it flexes as the legs 536 are wrapped around a limb. The shield portion 532 has an edge portion, between the elongate legs 536, which includes a number of independently moveable sections 540 on its outer edge which are able to move relative to one another. The combination of the separated sections 540 with a flexible material allows the shield portion 532 to flex into a three-dimensional configuration without the need for any hinge line(s).

FIG. 27a shows an alternative embodiment of an integrally formed shielding device 530′ which also does not have hinge lines. In this embodiment, the independently moveable sections 540′ along the outer edge are longer than those in FIG. 26. This creates a different three-dimensional shape when the shield portion 532′ is flexed and the legs 536 wrapped around a limb, as demonstrated in FIGS. 27b-27d. The moveable sections 540′ remove the need for a hinge line, providing an effective hinged region as they move to adapt to the limb. By changing the size and number of the moveable sections 540′, the standing configuration of the shield portion 532′ can be adapted. This can be used to create shielding devices 530′ which are designed for use on a particular limb, e.g. arranged such they stand at an angle appropriate for their use.

Although the shielding devices of the various embodiments illustrated herein are generally shown to be plain, it will be understood that one or more of their surfaces may carry graphics and/or text as desired. The shielding devices may be formed from a material, e.g. card, paper or plastic, that can be printed upon during manufacture.

It should be apparent that the foregoing relates only to the preferred embodiments of the present application and the resultant patent. Numerous changes and modification may be made herein by one of ordinary skill in the art without departing from the general spirit and scope of the invention as defined by the following claims and the equivalents thereof.

We claim:

1.-62. (canceled)

63. A device for shielding at least a portion of a limb from the view of a wearer of the device in use, the device comprising: a shield part, an adaption part and an attachment part; wherein:
the shield part comprises an upper portion and a lower portion;
the attachment part comprises at least one elongate member extending away from a lower portion of the shield part;
the adaption part is connected to a lower portion of the shield part;
the device has a periphery that runs continuously along an outer edge of the adaption part, along an outer edge of the at least one elongate member and along an outer edge of the upper portion of the shield part; and
the outer edge of the adaption part is defined by a plurality of cuts or cut-away portions extending into the adaption part towards the lower portion of the shield part to form a plurality of independently moveable sections arranged next to each other along the periphery which are free to move with respect to one another to thereby adapt to a shape of a limb in contact with the adaption part in use;
the device being formed from a single sheet of flexible material; and
the device having a first configuration in which the periphery extends around the sheet of flexible material in a single plane and a second configuration in which the periphery extends around the sheet of flexible material in multiple planes, one or more of the plurality of independently moveable sections flexing relative to the shield part and relative to one another to adapt to a shape of a limb in contact with the adaption part and thereby change, in use, how the periphery extends in the multiple planes to define a three-dimensional shape for the device in the second configuration.

64. A device according to claim 63, wherein the plurality of cut-away portions are arranged such that the plurality of independently moveable sections are spaced apart with respect to one another.

65. A device according to claim 63, wherein the adaption part has an outer profile extending from one independently moveable section to the next and each of the plurality of cuts or cut-away portions extends substantially orthogonal to a tangent to the profile at the location of the cut or cut-away portion.

66. A device according to claim 63, wherein the adaption part has a curved outer profile extending around the plurality of independently moveable sections.

67. A device according to claim 63, wherein the plurality of cuts or cut-away portions are spaced substantially equally along an outer profile of the adaption part.

68. A device according to claim 63, wherein the at least one elongate member extending away from the lower portion of the shield part extends in a direction away from the upper portion.

69. A device according to claim 63, wherein the at least one elongate member comprises first and second elongate members each extending away from the lower portion of the shield part.

70. A device according, to claim 69, wherein the adaption part is connected to the lower portion of the shield part between the first and second elongate members.

71. A device according to claim 63, wherein the attachment part is adjustable.

72. A device according to claim 69, wherein at least one of the first and second elongate members adjustably fastens to another of the first and second elongate members.

73. A device according to claim 63, wherein in the second configuration the device is self-supporting.

74. A device according to claim 63, wherein the shield part comprises one or more hinge lines to assist in converting the device from the first configuration to the second configuration.

75. A device according to claim 63, wherein the adaption part is connected to the lower portion of the shield part by a hinge line.

76. A device according to claim 75, wherein the hinge line follows a curved path.

77. A device according to claim 75, wherein the hinge line is a line of weakness.

78. A device according to claim 63, where the attachment part or shield part includes an adhesive panel.

79. A device according to claim 63, comprising at least one point of weakness to enable the shield part to be separated from the attachment part after use.

80. A device according to claim 63, wherein the single sheet of flexible material is made from one or more of paper, card, plastic or plastic laminate.

81. A device according to claim 63, wherein the device is includes at least one adhesive patch that is removable from the device.
